(19) United States
(12) Patent Application Publication
(10) Pub. No.: US 2012/0288481 A1
(43) Pub. Date: Nov. 15, 2012

(54) TREATMENT OF HEART DISEASE

(75) Inventors: Piero Anversa, Boston, MA (US); Jan Kajstura, Brookline, MA (US); Annarosa Leri, Boston, MA (US); Polina Goihberg, Brookline, MA (US)

(73) Assignee: THE BRIGHAM AND WOMEN'S HOSPITAL, INC., Boston, MA (US)

(21) Appl. No.: 13/508,819

(22) PCT Filed: Nov. 9, 2010
(86) PCT No.: PCT/US10/55999

§ 371 Date: Aug. 1, 2012

Related U.S. Application Data
(60) Provisional application No. 61/259,357, filed on Nov. 9, 2009.

(51) Int. Cl. A61K 35/34; A61P 9/10; A61P 9/00

(52) U.S. Cl. 424/93.7

(57) ABSTRACT

Disclosed herein are methods, compositions and kits for treating cardiac stem cells to be administered to a subject in need thereof, e.g., with a damaged myocardium. The methods, composition and kits of the invention can be used to treat cardiovascular diseases such as heart failure, myocardial infarction and an age-related cardiomyopathy.

### CROSS-REFERENCE TO RELATED APPLICATIONS

This International application paragraphs the benefit of priority under 35 U.S.C. §119(e) of U.S. Provisional Application Nos. 61/259,357, filed Nov. 9, 2009, the contents of each of which are incorporated herein by reference in their entirety.

### GOVERNMENT SUPPORT

This invention was made with government support under Grant No. P01HL092868 awarded by National Institute of Health (NIH)/National Heart, Lung and Blood Institute (NHLBI). The government has certain rights in the invention.

### FIELD OF THE INVENTION

The invention relates generally to methods, compositions and kits for treatment of heart disease, and more particularly relates to methods, compositions and kits comprising cardiac stem cells for repairing a damaged heart tissue.

### BACKGROUND OF THE INVENTION

Cardiovascular disease is a major health risk throughout the industrialized world. An estimated 81.1 million Americans suffer from one or more types of cardiovascular disease, including high blood pressure, coronary heart disease, heart failure, and stroke (Heart Disease and Stroke Statistics, American Heart Association, 2010). Cardiovascular disease is one of the leading causes of death in Americans.

Among cardiovascular diseases, ischemic heart disease is the most common cause of death in most western countries. Ischemic heart disease is characterized chronically by a healed infarct, foci of myocardial scarring, cavitary dilation, and impaired ventricular performance. One serious condition is myocardial infarction (MI), commonly known as a heart attack. Estimates for 2006 show that 8.5 million people in the United States suffer from MI (Heart Disease and Stroke Statistics, American Heart Association, 2010). MI is caused by a sudden and sustained lack of blood flow to an area of the heart, typically caused by narrowing of a coronary artery. Without adequate blood supply, the tissue becomes ischemic, leading to the death of myocytes and vascular structures. This area of necrotic tissue is referred to as the infarct site, the size of which determines survival, with the probability of recovery decreasing with increasing infarct size. For example, in humans, an infarct of 46% or more of the left ventricle triggers irreversible cardiogenic shock and death.

Although an ischemic injury can initiate a healing process, it leads to formation of a scar that does not possess the biochemical, physical and functional properties of the original myocardial tissue, and therefore, negatively affects the overall performance of the heart. These myocardial alterations can only be reversed by replacement of scarred tissue with functionally competent myocardium. Leri A et al, 85 Physiol Rev. 1373 (2005).

There is mounting evidence to suggest that the heart has regenerative potential in the event of myocardial injury. Recent studies have shown that a resident population of cardiac stem cells (CSCs) in the heart contains both vasculogenic and myogenic lineages. Thus, regeneration of myocardial tissue after acute infarction has been attempted by employing CSCs. For example, implantation of CSCs to the damaged myocardium or local activation of resident CSCs by intramyocardial administration of growth factors has been recently shown to have a possibility of recovering ventricular muscle mass in an in vivo model. Linke A et al., 102 PNAS 8966 (2005); Urbanek K et al., 97 Circ Res. 663 (2005); Rota M et al., 103 Circ Res. 107 (2008). Yet one of the major factors responsible for successful implementation of cell therapy in the diseased heart involves the migratory ability of the delivered cardiac stem cells (CSCs). Only a small fraction of the CSCs are able to migrate to the damaged myocardium. In addition, aging can attenuate CSC locomotion, reducing cell replacement and the substitution of damaged cells and vascular structures. As such, there is a strong need to develop methods for facilitating CSC migration to damaged myocardium. Such methods would significantly improve stem cell-mediated treatment of heart diseases, e.g., myocardial infarctions, and provide new approaches to the management of human heart failure.

### SUMMARY OF THE INVENTION

Aspects of the present invention stem from the discovery that human cardiac stem cells (hCSCs) express EphA2 receptors while myocytes adjacent thereto express ephrin A1 ligand. Further, it was discovered that activation of hCSCs with ephrin A1, for example, pre-treatment of hCSCs with ephrin A1 prior to delivery to the border zone of an infarcted heart, promotes hCSC translocation to an infarcted myocardium and thus enhances myocardial regeneration in an in vivo mouse model.

Accordingly, provided herein are methods, compositions and kits for treating cardiac stem cells to be administered to a subject in need thereof. Examples of the subject in need thereof include, but are not limited to an individual diagnosed with or suffering from a myocardial infarction, a heart failure or an age-related cardiomyopathy. In one aspect, the method includes (a) contacting a population of cardiac stem cells with an effective amount of at least one EphA2 receptor agonist; and (b) administering the population of cardiac stem cells from step (a) to the subject in need thereof. In one embodiment, the at least one cardiac stem cell translocates to an area of a damaged heart tissue in the subject after administration.

In one embodiment, the population of cardiac stem cells is preselected for expression of EphA2 receptor, e.g., ˜100% of the population expresses the EphA2 receptor.

In one embodiment, the effective amount of at least one EphA2 receptor agonist is sufficient to increase motility of the cardiac stem cells by at least about 10%, as compared to cardiac stem cells in the absence of an EphA2 receptor agonist. In another embodiment, the effective amount of at least one EphA2 receptor agonist is sufficient to change structure of actin cytoskeleton from a sessile to a motile state, as compared to cardiac stem cells in the absence of an EphA2 receptor agonist.

In one embodiment, the effective amount of at least one EphA2 receptor agonist is sufficient to increase the locomotion speed of at least one cardiac stem cell within a myocardium by about 2-fold.

In one embodiment, the effective amount of at least one EphA2 receptor is about 50 ng/ml to about 20 μg/mL. In one embodiment, the effective amount of at least one EphA2 receptor is about 200 ng/mL to about 1 μg/mL.

In various embodiments, the EphA2 receptor agonist is ephrin A1 or a variant thereof. In one embodiment, the effective amount of ephrin A1 is 50 ng/mL to 20 μg/mL. In one embodiment, the effective amount of ephrin A1 is about 200 ng/mL to about 1 μg/mL.

In one embodiment, the EphA2 receptor agonist is recombinant ephrin A1-Fc or a variant thereof. In one embodiment, the effective amount of ephrin A1-Fc is about 50 ng/mL to 20 μg/mL. In one embodiment, the effective amount of ephrin A1-Fc is about 200 ng/mL to about 1 μg/mL.

After contacting the population of cardiac stem cells with at least one EphA2 receptor agonist, e.g., ephrin A1 or a variant thereof, for a pre-defined amount of time, e.g., about 5 to about 30 minutes, or in one embodiment, about 15 minutes, the cardiac stem cells are administered to a subject in need thereof, e.g., by injection or a catheter. In one embodiment, the cardiac stem cells are administered in spatial proximity to an area of the damaged heart tissue of the subject. In another embodiment, the cardiac stem cells are administered to the border of an area of the damaged heart tissue of the subject.

In various embodiments, the cardiac stem cells can be isolated from a myocardial tissue of a subject.

Another aspect of the invention provides a composition that comprises at least one cardiac stem cell and an effective amount of at least one EphA2 receptor agonist, e.g., ephrin A1 or a variant thereof. In one embodiment, the composition further comprises a pharmaceutically acceptable carrier. In another embodiment, the composition further comprises a cell culture medium.

In some embodiments, the composition disclosed herein comprises an amount of at least one EphA2 receptor agonist, e.g., ephrin A1 or a variant thereof, effective to increase motility of the cardiac stem cells by at least about 10%, as compared to cardiac stem cells in the absence of an EphA2 receptor agonist. In another embodiment, the composition disclosed herein comprises an amount of at least one EphA2 receptor agonist effective to change structure of actin cytoskeleton from a sessile to a motile state, as compared to cardiac stem cells in the absence of an EphA2 receptor agonist.

The composition disclosed herein is administered to a subject in need thereof. Examples of the subject in need thereof include, but are not limited to an individual diagnosed with or suffering from a myocardial infarction, a heart failure or an age-related cardiomyopathy. In various embodiments, the subject in need thereof is a mammal, e.g., a human.

In some embodiments, the composition disclosed herein is administered in spatial proximity to an area of a damaged heart tissue of the subject. In another embodiment, the composition of the invention is administered to the border of an area of a damaged heart tissue of the subject. Exemplary methods of administering cardiac stem cells to the subject include, but are not limited to, injection or delivery by a catheter.

Kits useful in carrying out the methods described herein also are provided. Such kits comprise at least one cardiac stem cell and an effective amount of at least one EphA2 receptor agonist, e.g., ephrin A1 or a variant thereof. In one embodiment, the kit disclosed herein comprises the composition of the invention. In various embodiments, the kit can optionally contain instructions for using the kit to carry out the methods described herein. In certain embodiment, the kit of the invention further comprises at least one syringe, one container and/or one catheter.

### DETAILED DESCRIPTION OF THE INVENTION

Ischemic heart disease is a disease characterized by ischemia (reduced blood flow) to the heart tissue, usually due to coronary artery disease. One of the serious conditions is myocardial infarction (MI), commonly known as a heart attack. Due to an inadequate blood supply, the heart tissue becomes necrotic, which is known as an infarct. The size of the infarct negatively affects an individual's survival or recovery probability. There have been a few attempts to regenerate the necrotic tissue by transplanting cardiac stem cells to the proximity of the damaged myocardium. However, only a few cardiac stem cells translocate to the damaged myocardium. Thus, stem cell therapy for restoration of a damaged myocardium to its original condition has not yet been identified.

In accordance with aspects of the invention, EphA2 receptor expression has been documented in cardiac stem cells, which in turn facilitates motility of cardiac stem cells after activation with an EphA2 receptor agonist, e.g., ephrin A1 or ephrin A1-Fc. The inventors have demonstrated that pre-treatment of cardiac stem cells with an EphA1 receptor agonist, e.g., ephrin A1 or ephrin A1-Fc, prior to injection into infarcted mice, promotes translocation of cardiac stem cells to the damaged myocardium and thus enhances myocardium regeneration.

Accordingly, some embodiments of the invention are generally related to methods, compositions and kits for treating cardiac stem cells to be administered to a subject in need thereof, e.g., an individual diagnosed with or suffering from a damaged myocardium. Another aspect of the invention relates to methods, compositions and kits for increasing motility of cardiac stem cells to be administered to a subject in need thereof. A further aspect of the invention is directed to methods, compositions and kits for therapeutic treatment of heart diseases, e.g., myocardial infarction, heart failure or an age-related cardiomyopathy.

One aspect of the invention provides a method for treating cardiac stem cells to be administered to a subject in need thereof. The method includes (a) contacting a population of cardiac stem cells with an effective amount of at least one EphA2 receptor agonist; and (b) administering the population of cardiac stem cells from step (a) to the subject in need thereof. In one embodiment, at least one cardiac stem cell translocates to an area of a damaged heart tissue in the subject after the administration.

As used herein, the term “contacting” refers to any suitable means for delivering, or exposing, an agent to at least one cell. Exemplary delivery methods include, but are not limited to, direct delivery to cell culture medium, delivery to an in vitro scaffold in which cells are seeded, e.g., via perfusion or injection, or other delivery method well known to one skilled in the art. In one embodiment, an EphA2 receptor agonist is added to the cell culture medium in which cardiac stem cells are cultured. In another embodiment, an EphA2 receptor agonist is coated on a solid support on which the cardiac stem cells are attached. In still another embodiment, an EphA2 receptor agonist is injected into a biocompatible gel (e.g., peptide gel, hydrogel) in which cardiac stem cells are encapsulated. In one embodiment, a population of cardiac stem cells is contacted with EphA2 receptor agonist added to the cell culture medium. The term “treatment” or “treated” in reference to exposing cells to an agent, e.g., treatment of cardiac stem cells with an EphA2 receptor agonist, is used herein interchangeably with the term “contacting”.

The cardiac stem cells can be contacted with at least one EphA2 receptor agonist for any period of time, e.g., minutes, hours, or days. In some embodiments, the population of cardiac stem cells described herein are contacted with at least one EphA2 receptor agonist for about 5 to about 30 minutes, for about 30 minutes to about 2 hours, for about 2 hours to about 6 hours, for about 6 hours to about 12 hours, for about 1 day to 2 days, for about 2 days to about 1 week, for about 1 week to about 1 month. In one embodiment, the cardiac stem cells are contacted with at least one EphA2 receptor agonist for at least about 1 minute, at least about 5 minutes, at least about 10 minutes or at least about 15 minutes. In one embodiment, the cardiac stem cells are contacted with at least one EphA2 receptor agonist for about 15 minutes.

In some embodiments, the population of cardiac stem cells described herein is contacted more than once with at least one EphA2 receptor agonist. In some embodiments, the cardiac stem cells can be contacted with one or more EphA2 receptor agonist at least twice, at least three times, at least four times, or at least five times. A different EphA2 receptor agonist or a combination thereof can be used in each cell treatment.

In some embodiments, the cardiac stem cells can be contacted with at least one additional cytokine prior to administration, such as hepatocyte growth factor (HGF), insulin-like growth factor-1 (IGF-1), or a variant thereof. In one embodiment, cardiac stem cells are contacted with at least one additional cytokine prior to contacting with an EphA2 receptor agonist. In alternative embodiment, cardiac stem cells are contacted with at least one additional cytokine after treatment with an EphA2 receptor agonist. In such embodiments, the EphA2 agonist-treated cardiac stem cells can be contacted with the additional cytokine prior to administration to a subject in need thereof.

In one embodiment, the cardiac stem cells are optionally contacted with hepatocyte growth factor (HGF) and/or insulin-like growth factor-1 (IGF-1). In such an embodiment, HGF can be present in an amount of about 0.1 ng/ml to about 400 ng/ml. In another embodiment, HGF can be present in an amount of about 25 ng/ml, about 50 ng/ml, about 75 ng/ml, about 100 ng/ml, about 125 ng/ml, about 150 ng/ml, about 175 ng/ml, about 200 ng/ml, about 225 ng/ml, about 250 ng/ml, about 275 ng/ml, about 300 ng/ml, about 325 ng/ml, about 350 ng/ml, about 375 ng/ml or about 400 ng/ml. In some embodiments, HGF can be present in an amount of at least about 25 ng/ml, at least about 100 ng/mL or at least about 200 ng/mL. In another embodiment, IGF-1 can be present in an amount of about 0.1 ng/ml to about 500 ng/ml. In yet a further embodiment, IGF-1 can be present in an amount of about 25 ng/ml, about 50 ng/ml, about 75 ng/ml, about 100 ng/ml, about 125 ng/ml, about 150 ng/ml, about 175 ng/ml, about 200 ng/ml, about 225 ng/ml, about 250 ng/ml, about 275 ng/ml, about 300 ng/ml, about 325 ng/ml, about 350 ng/ml, about 375 ng/ml, about 400 ng/ml, about 425 ng/ml, about 450 ng/ml, about 475 ng/ml, or about 500 ng/ml. In some embodiments, IGF-1 can be present in an amount of at least about 25 ng/ml, at least about 100 ng/mL or at least about 200 ng/mL. HGF positively influences stem cell migration and homing through the activation of the c-Met receptor (Kollet et al. (2003) J. Clin. Invest. 112: 160-169; Linke et al. (2005) Proc. Natl. Acad. Sci. USA 102: 8966-8971; Rosu-Myles et al. (2005) J. Cell. Sci. 118: 4343-4352; Urbanek et al. (2005) Circ. Res. 97: 663-673). Similarly, IGF-1 and its corresponding receptor (IGF-1R) induce cardiac stem cell division, upregulate telomerase activity, hinder replicative senescence and preserve the pool of functionally-competent cardiac stem cells in the heart (Kajstura et al. (2001) Diabetes 50: 1414-1424; Torella et al. (2004) Circ. Res. 94: 514-524; Davis et al. (2006) Proc. Natl. Acad. Sci. USA 103: 8155-8160).

Some other non-limiting examples of cytokines that can be used to optionally treat cardiac stem cells prior to administration include Activin A, Bone Morphogenic Protein 2, Bone Morphogenic Protein 4, Bone Morphogenic Protein 6, Cardiotrophin-1, Fibroblast Growth Factor 1, Fibroblast Growth Factor 4, Flt3 Ligand, Glial-Derived Neurotrophic Factor, Heparin, Insulin-like Growth Factor-2, Insulin-Like Growth Factor Binding Protein-3, Insulin-Like Growth Factor Binding Protein-5, Interleukin-3, Interleukin-6, Interleukin-8, Leukemia Inhibitory Factor, Midkine, Platelet-Derived Growth Factor AA, Platelet-Derived Growth Factor BB, Progesterone, Putrescine, Stem Cell Factor, Stromal-Derived Factor-1, Thrombopoietin, Transforming Growth Factor-α, Transforming Growth Factor-β1, Transforming Growth Factor-P2, Transforming Growth Factor-β3, Vascular Endothelial Growth Factor, Wnt1, Wnt3a, and Wnt5a, and variants thereof, as described in Kanemura et al. (2005) Cell Transplant. 14:673-682; Kaplan et al. (2005) Nature 438:750-751; Xu et al. (2005) Methods Mol. Med. 121:189-202; Quinn et al. (2005) Methods Mol. Med. 121:125-148; Almeida et al. (2005) J. Biol. Chem. 280:41342-41351; Bamabe-Heider et al (2005) Neuron 48:253-265; Madlambayan et al. (2005) Exp Hematol 33: 1229-1239; Kamanga-Sollo et al. (2005) Exp Cell Res 311:167-176; Heese et al. (2005) Neuro-oncol. 7:476-484; He et al. (2005) Am J. Physiol. 289:H968-H972; Beattie et al. (2005) Stem Cells 23:489-495; Sekiya et al. (2005) Cell Tissue Res 320:269-276; Weidt (2004) Stem Cells 22:890-896; Encabo et al (2004) Stem Cells 22:725-740; and Buytaeri-Hoefen et al. (2004) Stem Cells 22:669-674, the entire text of each of which is incorporated herein by reference.

The cardiac stem cells, after treatment with at least one EphA2 receptor agonist and at least one optional cytokine described above (referred herein as treated cardiac stem cells), can be administered to a subject in need thereof. Modes of administration of cells to a heart tissue are well known to those of skill in the art. In methods of the invention, any suitable mode can be used, e.g., injection, implantation, catheterization and intracoronary administration. In one embodiment, the treated cardiac stem cells are administered in spatial proximity to an area of a damaged heart tissue of the subject. In another embodiment, the treated cardiac stem cells are administered to the border of an area of a damaged heart tissue of the subject. As used herein “damaged heart tissue” refers to heart tissue cells which have been exposed to ischemic conditions, i.e. a condition characterized by a lack of oxygen supply to tissues of an organ due to inadequate perfusion. These ischemic conditions may be caused by a myocardial infarction, or other cardiovascular disease or related complaint. The lack of oxygen causes the death of the cells in the surrounding area, leaving an infarct, which will eventually scar. The terms “damaged heart tissue” and “damaged myocardium” have been used herein interchangeably.

The treated cardiac stem cells can be delivered to the heart by one or more administrations. In one embodiment, the treated cardiac stem cells are delivered by a single administration. In another embodiment, multiple administrations of the same or different populations of treated cardiac stem cells are delivered to the heart.

In some embodiments, administration of the treated cardiac stem cells to a subject in need thereof can be accompanied by the administration of one or more agent, e.g., an EphA2 receptor agonist or a cytokine, to the heart. Non-limiting examples of cytokines that can be administered include: stem cell factor (SCF), granulocyte-colony stimulating factor (G-CSF), granulocyte-macrophage colony stimulating factor (GM-CSF), stromal cell-derived factor-1, steel factor, vascular endothelial growth factor, macrophage colony stimulating factor, granulocyte-macrophage stimulating factor, hepatocyte growth factor (HGF), insulin-like growth factor-1 (IGF-1), Interleukin-3, or any cytokine capable of the stimulating and/or mobilizing stem cells. In one embodiment, the EphA2 receptor agonist is ephrin A1, or a variant thereof. In another embodiment, the EphA2 receptor agonist is recombinant ephrin A1-Fc, or a variant thereof. In one embodiment, the cytokines are selected from HGF, IGF-1, functional variants of HGF or IGF-1, or combinations thereof.

In embodiments of the invention, at least one EphA2 receptor agonist (e.g., ephrin A1 or a variant thereof) and/or at least one additional cytokine (e.g., HGF and/or IGF-1) can be delivered simultaneously with the treated cardiac stem cells. Alternatively, the administration of the EphA2 receptor agonists and/or additional cytokines can either precede or follow the administration of the treated cardiac stem cells by a specified time period. The time period can be about 5 minutes, about 15 minutes, about 30 minutes, about 1 hour, about 3 hours, about 6 hours, about 12 hours, about 24 hours, about 36 hours, about 1 week, about 2 weeks, about 1 month, about 6 months or about 1 year. In some embodiments, the time period can be at least about 1 minute, at least about 5 minutes, at least about 15 minutes, at least about 30 minutes, at least about 1 hour, at least about 6 hours, at least about 1 day, at least about 1 week, at least about 1 month, or at least about 6 months.

The treated cardiac stem cells and/or an agent such as EphA2 agonist and cytokine can be administered to the heart of the subject in need thereof by injection. In one embodiment, the injection is intramyocardial. One of skill in the art would be well aware of advantages of delivering cardiac stem cells by intramyocardial injection as the heart is a functioning muscle. Intramyocardial injection minimizes the loss of the injected cardiac stem cells due to the contracting movements of the heart. Alternatively, the treated cardiac stem cells can be administered by injection transendocardially or trans-epicardially. This mode of injection allows the cytokines to penetrate the protective surrounding membrane. In one embodiment, a catheter-based approach is used to deliver the trans-endocardial injection. The use of a catheter precludes more invasive methods of delivery wherein the opening of the chest cavity would be necessitated. As one skilled in the art would appreciate, optimum time of recovery would be allowed by the more minimally invasive procedure. A catheter approach can involve the use of such techniques as the NOGA catheter or similar systems. The NOGA catheter system facilitates guided administration by providing electromechanic mapping of the area of interest, as well as a retractable needle that can be used to deliver targeted injections or to bathe a targeted area with a therapeutic. Any methods of the invention can be performed through the use of such a system to deliver injections. One of skill in the art will recognize alternate systems that also provide the ability to provide targeted treatment through the integration of imaging and a catheter delivery system that can be used with the methods of the invention. Information regarding the use of NOGA and similar systems can be found in, for example, Sherman (2003) Basic Appl. Myol. 13: 11-14; Patel et al (2005) The Journal of Thoracic and Cardiovascular Surgery 130:1631-38; and Perrin et al. (2003) Circulation 107: 2294-2302; the text of each of which are incorporated herein in their entirety. In another embodiment, the cardiac stem cells can be administered by an intracoronary route of administration. One of skill in the art will recognize other useful methods of delivery or implantation which can be utilized with the methods of the invention, including those described in Dawn et al. (2005) Proc. Natl. Acad. Sci. USA 102, 3766-3771, the contents of which are incorporated herein in their entirety.

In embodiments of the invention, administration of treated cardiac stem cells can result in amelioration of at least one symptom associated with cardiovascular disease, e.g., heart failure, myocardial infarction, an age-related cardiomyopathy or a damaged myocardium. In one embodiment, at least one symptom of the cardiovascular disease is alleviated by at least about 10%, at least about 15%, at least about 20%, at least about 30%, at least about 40%, or at least about 50%. In one embodiment, at least one symptom is alleviated by more than 50%. In one embodiment, at least one symptom is alleviated by at least about 80%, at least about 90% or greater, as compared to the severity of symptoms in the absence of administration with treated hCSCs.

In another aspect of the invention, migration of resident cardiac stem cells (i.e., endogenous cardiac stem cells that reside within the heart of a subject) to a damaged heart tissue of the subject can be enhanced by administering (e.g., by intramyocardial injection) an effective amount of at least one EphA2 receptor agonist to the subject in need thereof. Accordingly, in one embodiment, an effective amount of at least one EphA2 receptor agonist is administered to a subject, wherein the subject is suspected of having a cardiovascular disease, or is diagnosed with or suffering from a cardiovascular disease. In one embodiment, the subject is diagnosed with or suffering from a myocardial infarction.

In one embodiment, the subject is administered with an effective amount of EphA2 receptor agonist sufficient to increase migration of resident cardiac stem cells by at least about 10%, at least about 20%, at least about 30%, at least about 40%, at least about 50%, at least about 60%, at least about 70%, at least about 80%, at least about 90%, about 95%, about 98%, about 99% or 100%, as compared to cardiac stem cells in the absence of an EphA2 receptor agonist. In another embodiment, the effective amount of EphA2 receptor agonist is an amount sufficient to increase migration of at least one cardiac stem cell within a myocardium of the subject by at least about 1.5-fold or at least about 2-fold. In one embodiment, the effective amount of EphA2 receptor agonist is an amount sufficient to increase migration of at least one cardiac stem cell by about 2-fold. In vivo migration within a myocardium can be assessed by echocardiography, which has been described in U.S. Pat. No. 7,547,674, the content of which is incorporated herein by reference in its entirety.

In one embodiment, the EphA2 receptor agonist is ephrin A1, or a variant thereof, e.g., ephrin A1-Fc. In such embodiment, the effective amount of ephrin A1, or a variant thereof, e.g., ephrin A1-Fc, for in vivo administration is about 0.1 μg/ml to about 100 μg/ml. In some embodiments, the effective amount of ephrin A1, or a variant thereof, e.g., ephrin A1-Fc, for in vivo administration can be present in an amount of about 0.2 μg/ml, about 0.3 μg/ml, about 0.4 μg/ml, about 0.5 μg/ml, about 0.6 μg/ml, about 0.7 μg/ml, about 0.8 μg/ml, about 0.9 μg/ml, about 1 μg/ml, about 1.5 μg/ml, about 2 μg/ml, about 2.5 μg/ml, about 3 μg/ml, about 3.5 μg/ml, about 4 μg/ml, about 4.5 μg/ml, about 5 μg/ml, about 5.5 μg/ml, about 6 μg/ml, about 6.5 μg/ml, about 7 μg/ml, about 7.5 μg/ml, about 8 μg/ml, about 8.5 μg/ml, about 9 μg/ml, about 9.5 μg/ml, about 10 μg/ml, about 20 μg/ml, about 30 μg/ml, about 40 μg/ml, about 50 μg/ml, about 60 μg/ml, about 70 μg/ml, about 80 μg/ml, about 90 μg/ml or about 100 μg/ml. In some embodiments, the effective amount of ephrin A1, or a variant thereof, e.g., ephrin A1-Fc, for in vivo administration can be present in an amount of at least about 0.1 μg/ml, at least about 0.5 μg/ml, at least about 1 μg/ml, at least about 10 μg/ml, at least about 25 μg/ml, or at least about 50 μg/ml.

In one embodiment, effective amount of ephrin A1, or a variant thereof, e.g., ephrin A1-Fc, for in vivo administration is about 100 μg/ml to about 1000 μg/ml. In some embodiments, the effective amount of ephrin A1, or a variant thereof, e.g., ephrin A1-Fc, for in vivo administration can be present in an amount of about 100 μg/ml, about 150 μg/ml, about 200 μg/ml, about 250 μg/ml, about 300 μg/ml, about 350 μg/ml, about 400 μg/ml, about 450 μg/ml, about 500 μg/ml, about 550 μg/ml, about 600 μg/ml, about 650 μg/ml, about 700 μg/ml, about 750 μg/ml, about 800 μg/ml, about 850 μg/ml, about 900 μg/ml, about 950 μg/ml, or about 1000 μg/ml.

Cardiac Stem Cells

The term “cardiac stem cells” as used herein refer to cardiac cells that have the ability to renew themselves through mitosis as well as the ability to differentiate into more than one specialized cell type, such as cardiomyocytes, smooth muscle cells and endothelial cells, i.e. the cells are multipotent. In one embodiment, the cardiac stem cells express c-kit. In one embodiment, the cardiac stem cells express c-kit and are negative for hematopoietic markers including CD34, CD45, CD133, CD105, CD90 and multiple markers of bone marrow cell lineages. In one embodiment, cardiac stem cells are negative for cardiac transcription factors (e.g., Nkx2.5, MEF2C, GATA4, GATA6, Etsl) and cardiac cytoplasmic/membrane proteins (e.g. α-sarcomeric actin, α-smooth muscle actin, vWf, CD31). In some embodiments, cardiac stem cells are not precursors or progenitors although during differentiation they acquire markers of cardiovascular lineages.

In one embodiment, the cardiac stem cells are somatic stem cells. The term “somatic stem cells” as used herein generally refers to multipotent stem cells that are not derived from the germline (e.g., sperms or eggs) and that can differentiate into more than one cell type of an organ from which they originate, e.g., heart. In one embodiment, the somatic stem cells are stem cells derived from a heart tissue.

In some embodiments of the invention, a population of cardiac stem cells can be isolated from a myocardial tissue of the subject, e.g., as described in Bearzi C et al., 104 PNAS14068 (2007). The term “population” as used herein refers to more than one cell with the same phenotypic characteristics. The phrase “a population of cardiac stem cells” as used herein refers to a collection of cells comprising more than one cardiac stem cell, e.g., at least about 10% cardiac stem cells, at least about 20% cardiac stem cells, at least about 30% cardiac stem cells, at least about 40% cardiac stem cells, at least about 50% cardiac stem cells, at least about 60% cardiac stem cells, at least about 70% cardiac stem cells, at least about 80% cardiac stem cells, at least about 90% cardiac stem cells, at least about 95%, about 98%, about 99% or 100% cardiac stem cells. Other cells that can be present in a population of cardiac stem cells can include, but are not limited to, cardiomyocytes, skeletal myoblasts, somatic stem cell, e.g., bone marrow stem cells, or any cells known in the art for supporting the survival and differentiation of cardiac stem cells to mature cardiomyocytes.

Methods for isolating cardiac stem cells are known in the art. Cardiac stem cells can be isolated from tissue specimens (e.g. myocardium or myocardial vessels) obtained from a subject or patient. By way of an example only, a myocardial tissue specimen can be minced and placed in appropriate culture medium. Cardiac stem cells growing out from the tissue specimens can be observed in approximately 1-2 weeks after initial culture. At approximately 4 weeks after the initial culture, the expanded stem cells can be collected by centrifugation. Other methods of isolating adult cardiac stem cells from a subject are known in the art and can be employed to obtain suitable stem cells for use in the methods of the invention. U.S. Patent Application Publication No. 2006/0239983, filed Feb. 16, 2006, which is incorporated herein by reference in its entirety, describes media appropriate for culturing and expanding adult stem cells, particularly human cardiac stem cells. However, one of ordinary skill in the art would be able to determine the necessary components and modify commonly used cell culture media to be employed in culturing the isolated cardiac stem cells that can be used in the methods, compositions and kits of the invention.

Cardiac stem cells (CSCs) can be obtained from a myocardial tissue of a subject. Methods for isolating and characterizing cardiac stem cells are described in U.S. Pat. App. Pub. No.: US 2009/0180998, US 2009/0148421, US 2009/0162329 and U.S. Pat. No. 7,547,674, the contents of which are incorporated herein by reference in their entirety. In the case of an autologous CSC donation, CSCs can be obtained from a fresh surgical sample, such as a cardiac biopsy performed for a clinical indication. The term “autologous” as used herein refers to an object that is derived or transferred from the same individual's body, e.g., autologous blood donation, autologous bone marrow transplant. In the case of an allogenic CSC donation, CSCs can be obtained from a surgical sample, such as a cardiac biopsy from a patient undergoing therapeutic transplantation or a donor heart not utilized for transplantation. As used herein, the term “allogeneic” refers to an object that is genetically different although belonging to or obtained from the same species, e.g., a human. The surgical sample or biopsy may be obtained from the right ventricle (RV), interventricular septum, left ventricle (LV), or any other region of the cardiac tissue that comprises cardiac stem cells. In one embodiment, the surgical sample is about 1 to about 5 grams in size. In another embodiment, the surgical sample is less than 1 gram in size. In some embodiments, CSCs can be found in regions of the atrium of a subject that are normally discarded during routine cardiac surgery. The cardiac stem cells described herein can be obtained by mechanically and enzymatically dissociating cells from human myocardial tissue present in the sample. Mechanical dissociation can be brought about using methods that include, without limitation, chopping and/or mincing the tissue, and/or centrifugation and the like. Enzymatic dissociation of connective tissue and from cell-to-cell associations can be brought about by enzymes including, but not limited to, Blendzyme, DNAse I, collegenase and trypsin, or a cocktail of enzymes found to be effective in liberating cardiac stem cells from the cardiac sample. The procedure for mechanically and enzymatically isolating a cardiac stem cell should not be construed to be limited to the materials and techniques presented herein, but rather it will be recognized that these techniques are well-established and fall well within the scope of experimental optimization performed routinely in the art.

In one embodiment, the cardiac stem cells described herein is lineage negative. The term “lineage negative” is known to one skilled in the art as a cell that does not express antigens characteristic of specific cell lineages. Lineage negative stem cells can be isolated by various means, including but not limited to, removing lineage positive cells by contacting a cell population with antibodies against lineage markers and subsequently isolating the antibody-bound cells by using an anti-immunoglobulin antibody conjugated to magnetic beads and a biomagnet. Alternatively, the antibody-bound lineage positive cells may be retained on a column containing beads conjugated to anti-immunoglobulin antibodies. The cells not bound to the immunomagnetic beads represent the lineage negative stem cell fraction and can be isolated. For instance, cells expressing markers of the cardiac lineage (e.g. markers of vascular cell or cardiomyocyte commitment) can be removed from the cell populations in order to isolate lineage negative cardiac stem cells. Markers of the vascular lineage include, but are not limited to, GATA6 (SMC transcription factor), Ets 1 (EC transcription factor), Tie-2 (angiopoietin receptors), VE-cadherin (cell adhesion molecule), CD62E/E-selectin (cell adhesion molecule), alpha-SM-actin (α-SMA, contractile protein), CD31 (PECAM-1), vWF (carrier of factor VIII), Bandeiraera simplicifolia and Ulex europaeus lectins (EC surface glycoprotein-binding molecules). Markers of the myocyte lineage include, but are not limited to, GATA4 (cardiac transcription factor), Nkx2.5 and MEF2C (myocyte transcription factors), and alpha-sarcomeric actin (a-SA, contractile protein).

In certain embodiments, the lineage negative cardiac stem cells express the stem cell surface marker, c-kit, which is the receptor for stem cell factor. Positive selection methods for isolating a population of lineage negative stem cells expressing c kit are well known to the skilled artisan. Examples of possible methods include, but are not limited to, various types of cell sorting, such as fluorescence activated cell sorting (FACS) and magnetic cell sorting as well as modified forms of affinity chromatography. In one embodiment, the lineage negative stem cells are c-kit positive.

In some embodiments of the invention, cardiac stem cells described herein express EphA2 receptor. The EphA2 receptor is a surface protein and can be detected by routine methods known to the skilled artisan to measure expression of surface markers. Such methods include, but are not limited to FACS, magnetic cell sorting, and modified forms of affinity chromatography. Alternatively, EphA2 receptor expression can be measured by immunocytochemistry or Western blotting techniques.

In some embodiments, the population of cardiac stem cells can further comprise vascular progenitors cells (VPCs) and myocyte progenitor cells (MPCs). Vascular progenitor cells can be isolated from a c-kit positive stem cell population, as described above, by selecting cells expressing the VEGFR2 receptor, flk1. Vascular progenitor cells are lineage negative, c-kit positive, and flk1 positive. Similarly, myocyte progenitor cells can be isolated from the c-kit positive stem cell population by selecting cells that do no express flk1. Myocyte progenitor cells are lineage negative, c-kit positive, and flk1 negative. Similar methods for isolating c-kit positive stem cells can be employed to select cells that express or do not express the flk1 receptor (e.g. immunobeads, cell sorting, affinity chromatography, etc.).

Isolated lineage negative, c-kit positive stem cells can be plated individually, for instance, in single wells of a cell culture plate, and further expanded to obtain clones from individual stem cells.

In one embodiment, telomere length is measured in the clones derived from single stem cells. Methods of determining telomere length are well known in the art. Telomere length may be assessed by using methods such as quantitative fluorescence in situ hybridization (Q-FISH), Southern Blot, or quantitative PCR. Cardiac stem cells with telomeres that are at least 5 kbp, at least 8 kbp, at least 10 kbp, at least 12 kbp, at least 13 kbp, at least 14 kbp, at least 15 kbp, at least 16 kbp, at least 17 kbp, or at least 18 kbp in length can be selected for use or further expansion in cell culture. In one embodiment, human cardiac stem cells with telomeres that are at least 5 kbp in length are selected for further use.

In another embodiment, telomerase activity is measured in the expanded cardiac stem cell clones. Methods of measuring telomerase activity can include electrophoretic and ELISA-based telomere repeat amplification protocol (TRAP) assays as well as real time PCR methods. Telomerase activity in the isolated cardiac stem cells can be compared to that in control cells. The control cells can be freshly isolated c-kit positive cardiac cells from young animals. In the case of human cardiac stem cells, the control cells can be freshly isolated c-kit positive cardiac cells from a young (20-40 years) individual. Cardiac stem cells expressing at least 60%, at least 70%, at least 80%, preferably 90%, or 95% of the telomerase activity as compared to control cells can be selected for use and further expansion.

In yet another embodiment, insulin-like growth factor-1 (IGF-1) receptor expression is assessed in the expanded cardiac stem cell clones. The IGF-1 receptor is a surface protein and can be detected by routine methods known to the skilled artisan to measure expression of surface markers. Such methods include, but are not limited to FACS, magnetic cell sorting, and modified forms of affinity chromatography. Alternatively, IGF-1 receptor expression can be measured by immunocytochemistry or Western blotting techniques. In one embodiment, cardiac stem cell clones positive for IGF-1 receptor expression are selected for further use.

In some embodiments, the expanded cardiac stem cells can further differentiate into more than one specific cell type, e.g., cardiomyocytes, endothelial cells or smooth muscle cell in vitro, by contacting them with at least one cardiac cytokine essential for cell differentiation. In some embodiments, at least about 20%, at least about 30%, at least about 40%, at least about 50%, at least about 60%, at least about 70%, at least about 80%, at least about 90%, at least about 95%, about 98%, about 99%, about 100% of the cardiac stem cells differentiate into cardiomyocytes, smooth muscle cells and/or endothelial cells in vitro.

In some embodiments, the cardiac stem cells can be derived from bone marrow stem cells or induced pluripotent stem cells of a subject, e.g., induced to have the characteristics of cardiac stem cells described herein. The bone marrow stem cells or induced pluripotent stem cells can be autologous or allogeneic.

EphA2 Receptor Agonist

The Eph family of receptors comprises fourteen structurally related transmembrane receptor tyrosine kinases and can be divided into two groups—EphA and EphB—on the basis of sequence homologies. Pasquale E. 9 Curr Opin Cell Biol. 608 (1997) and Orioli et al. 13 Trends in Genetics 354 (1997). EphA2 receptor, encoded by the EPHA2 gene, belongs to the ephrin receptor subfamily A of the protein-tyrosine kinase family, and it binds ephrin-A ligands. The EPHA2 gene is also known by other aliases ARCC2 or ECK, as well as designated as ephrin type-A receptor 2, epithelial cell receptor protein tyrosine kinase, soluble EPHA2 variant 1 or tyrosine-protein kinase receptor ECK. EPH and EPH-related receptors have been implicated in mediating developmental events, e.g., in the nervous system. Receptors in the EPH subfamily typically have a single kinase domain and an extracellular region containing a Cys-rich domain and 2 fibronectin type III repeats.

The term “agonist” as used herein refers to a molecule which is capable of activating one or more of the biological activities of a target molecule, such as an EphA2 receptor. Agonists may, for example, act by activating a target molecule and/or mediating signal transduction. EphA2 receptor agonist included within the scope of the invention are ephrin-A ligands (e.g., ephrin A1, ephrin A2, ephrin A3, ephrin A4, and ephrin A5), agonistic antibodies that bind to EphA2 receptor; amino acid sequence variants or derivatives of an ephrin ligand (e.g., ephrin A1) which activate the EphA2 receptor or Eph ligand; synthetic or native sequence peptides which bind to and activate EphA2 receptor; small molecule agonists; and a gene encoding ephrin ligand, e.g., ephrin A1 (i.e. for gene therapy).

In one embodiment, the EphA2 receptor agonist is ephrin A1 (also known in the art by aliases as EFNA1, B61, ECKLG, EFL1, EPLG1, LERK1, TNFAIP4). Other designations of ephrin A1 known in the art are LERK-1, TNF alpha-induced protein 4, eph-related receptor tyrosine kinase ligand 1, immediate early response protein B61, ligand of eph-related kinase 1, tumor necrosis factor alpha-induced protein 4, tumor necrosis factor and alpha-induced protein 4.

In another embodiment, the EphA2 receptor agonist can be a variant of ephrin A1. An exemplary variant of ephrin A1 is ephrinA1-Fc, the extracellular domain of ephrinAl linked to immunoglobulin heavy chain, (see Miao, H., et al., EphA2 kinase associates with focal adhesion kinase and upon activation, inhibits integrin-mediated cell adhesion and migration, Nature Cell Biol 2, 62-69 (2000), hereby incorporated by reference). Other variants of ephrin A1 are also included within the scope of the invention. For example, the amino acid sequence of ephrin A1 has been assigned a NCBI accession number for different species such as human, mouse and rat. In particular, the NCBI accession numbers for the amino acid sequences of human ephrin A1 are NP_{—}004419 (SEQ ID NO: 1) or NP_{—}872626 (SEQ ID NO: 2), each of which represents a different isoform. Further, the conserved domains of ephrin A1 in SEQ ID NO: 1 and SEQ ID NO: 2 are known to be located between aa17-152 and aa17-137, respectively. Thus, one of skill in the art can design a variant of ephrin A1, which can function as an EphA2 receptor agonist. In certain embodiments, an ephrin A1 variant has an amino acid sequence that is at least 70% identical to an amino acid sequence as set forth in SEQ ID NO: 1 or 2. In some embodiments, an ephrin A1 variant has an amino acid sequence at least 85%, 90%, 95%, 97%, 98%, 99% or 100% identical to an amino acid sequence as set forth in SEQ ID NO: 1 or 2.

In certain embodiments, an EphA2 receptor agonist can activate function of EphA2 receptor, activate the phosphorylation of EphA2 receptor, enhance dimerization of EphA2 receptor, or activate any of the downstream signaling events upon binding of an ephrin ligand (e.g., ephrin A1) to EphA2 receptor. For example, an EphA2 receptor agonist can be capable of binding to an EphA2 polypeptide (e.g., the ligand-binding domain of an EphA2 polypeptide) and function as an EphA2 ligand.

Generally, an EphA2 receptor agonist includes any molecules that act as agonists of EphA2 receptor. Such EphA2 receptor agonists include, but are not limited to, a protein, a peptide, a small organic molecule, a peptidomimetic, an agonistic antibody, and a nucleic acid. In some embodiments, these substances can comprise exogenous or non-native peptides or small molecules that have a molecular weight of about 50 daltons to about 2,500 daltons.

In some embodiments, an EphA2 receptor agonist can be a peptide, such as those which activate EphA2 kinase function. These peptides are also referred to herein as EphA2 agonistic peptides. These agonistic peptides can specifically target the ligand-binding domain of EphA2 kinase. In some embodiments, EphA2 agonistic peptides can comprise about 4 to about 20 amino acids and have a molecular weight of about 600 daltons to about 2,500 daltons. The amino acid sequence of EphA2 receptor has been assigned a NCBI accession number for different species such as human, mouse and rat. In particular, the NCBI accession number for the amino acid sequence of human EphA2 receptor are NP_{—}004422 (SEQ ID NO: 3). It is also known in the art that the ephrin receptor ligand binding domain is located between aa28-201 of SEQ ID NO.3. Further, the crystal structure of EphA2 ligand-binding domains has been described in Himanen J. P. et al., 10 EMBO Rep. 722 (2009). Thus, one of skill in the art can design agonistic peptides, proteins or variants thereof for activating EphA2 receptor.

In certain embodiments, the structure of an EphA agonistic peptide can be modified for such purposes as enhancing therapeutic efficacy, or stability (e.g., ex vivo shelf life or resistance to proteolytic degradation in vivo). Modified EphA2 agonistic peptides can be produced, for instance, by amino acid substitution, deletion, or addition. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of an EphA2 agonistic peptide results in a functional homolog can be readily determined by assessing the ability of the variant EphA2 agonistic peptide to produce a response (e.g., migration response measured by well-established trans-migration assay) in cardiac stem cells in a fashion similar to the wild-type EphA agonistic peptide.

In some embodiments, an EphA2 receptor agonist can be an agonistic antibody against EphA2 receptor, e.g., the ones disclosed in the U.S. Pat. App. Pub. No.: US 2007/0134254 and US 2010/0143345, the contents of which are incorporated herein by their entirety.

In some embodiments, an EphA2 receptor agonist can be a small molecule as disclosed in the PCT Application No.: WO 2009/008901, the content of which is incorporated herein by its entirety.

In some embodiments, EphA2 receptor agonist can be PIK3R1 [Pandey A. et al., 269 J. Biol. Chem. 30154 (1994)], Grb2 [Pratt R. L. et al., 21 Oncogene 7690 (2002)] ACP1 [Kikawa K. D. et al., 277 J. Biol. Chem. 39274 (2002)] and SHC1 [Pratt R. L. et al., 21 Oncogene 7690 (2002)].

In methods of the invention, cardiac stem cells are contacted with an effective amount of at least one EphA2 receptor agonist. Various established in vitro assays can be used to determine an effective amount of EphA2 receptor agonist for treating cardiac stem cells. For example, after contacting the population of cardiac stem cells with an amount of an EphA2 receptor agonist for a specific amount of time, a subset of the treated cells can be used for in vitro characterization, e.g., migration assay, while the rest of the treated cardiac stem cells can be used for administration to a subject in need thereof. The phrase “effective amount” as used herein refers to an amount of a compound, material, or composition which is effective for producing some desired effect in at least a sub-population of cells. For example, a population of cardiac stem cells is contacted with an amount of an EphA2 receptor agonist described herein sufficient to produce a statistically significant, measurable response as described in Example 3, when compared to cardiac stem cells in the absence of an EphA2 receptor agonist. An exemplary measurable response is migration of cardiac stem cells in response to a chemoattractant, e.g., HGF, which can be determined by a transwell migration assay well known in the art. During this assay, cells are placed on the upper surface of a cell permeable membrane and a solution containing a chemoattractant, e.g., HGF, is placed below the cell permeable membrane. Following a specific amount of time, e.g., about 3 to about 18 hours, the cells that have migrated through the membrane can be stained and counted, or lysed for total protein quantification. In accordance with aspects of the invention, treatment of cardiac stem cells with an EphA2 receptor agonist, e.g., ephrin A1 or ephrin A1-Fc, increases the number of cells migrating to the underside of the Transwell membrane in response to low concentrations of HGF. Thus, the number of cells that have migrated through the membrane or the total protein thereof (e.g., measured by established total protein assay) can be indicative of cell migration in response to a chemoattractant, e.g., HGF.

Accordingly, in one embodiment, the effective amount of an EphA2 receptor agonist is sufficient to increase the number of migrating cells in response to a chemoattractant, e.g., HGF, by at least about 10%, at least about 20%, at least about 30%, at least about 40%, at least about 50%, at least about 60%, at least about 70%, at least about 80%, at least about 90%, about 95%, about 98%, about 99% or 100%, as compared to cardiac stem cells in the absence of an EphA2 receptor agonist, e.g., measured by Transwell migration asaay.

Cell migration can also be determined by in vitro wound healing assay known to a skilled artisan. The basic steps of an in vitro wound healing assay involve creating a cell-devoid space (e.g., by making a scratch to remove some cells) in a cell monolayer, capturing the images at the beginning and at regular intervals during cell migration to close the wound, and comparing the images to quantify the migration rate of the cells. Accordingly, in one embodiment, the effective amount of EphA2 receptor is sufficient to increase the migration rate of cardiac stem cells by at least about 10%, at least about 20%, at least about 30%, at least about 40%, at least about 50%, at least about 60%, at least about 70%, at least about 80%, at least about 90%, about 95%, about 98%, about 99% or 100%, as compared to cardiac stem cells in the absence of an EphA2 receptor agonist. One of skill in the art is readily able to perform migration assays that are well established in the art, such as in vitro healing assays, and transwell migration assays. Commercial transwell migration assay kits (e.g., from Millipore) are also available for use in determining cell migration.

In some embodiments, the effective amount of EphA2 receptor agonist is sufficient to induce rearrangement of actin cytoskeleton of cardiac stem cells (and change cell shape of CSCs) from a sessile to a motile state, as compared to cardiac stem cells in the absence of an EphA2 receptor agonist. The phrase “sessile state” as used herein refers to a round shape, while the phrase “motile state” as used herein refers to an elongated and polarized shape. The term “polarized” as used herein refers to asymmetry in the organization of a cell. Motile cardiac stem cells are said to be polarized in the sense that motion requires the coordination of asymmetrical processes involving protrusion of their leading edges and retraction of their trailing edges. Methods for determining arrangement of actin cytoskeleton and/or cell shape are well known in the art, e.g., by immunostaining and imaging. For example, a subset of cardiac stem cells after treatment with an EphA2 receptor agonist can be examined for the change in cell shape by microscopy, e.g., brightview microscopy or phase-contrast microscopy. Alternatively, the treated cardiac stem cells can be stained with commercially available phalloidin or an actin antibody using well-known immunostaining protocols for detecting arrangement of actin cytoskeleton. One of skill in the art can readily distinguish sessile and motile states of a cell based on cell shape and/or actin arrangement.

As Src kinase is a well-known downstream effector of Eph receptor signaling, in one embodiment, the effective amount of EphA2 receptor agonist is determined by detecting an increase in phosphorylation of the activation site of Src kinase at tyrosine 416 and/or a decrease in phosphorylation of the inhibitory site of Src at tyrosine 527 by at least about 10%, at least about 20%, at least about 30%, at least about 40%, at least about 50%, at least about 60%, at least about 70%, at least about 80%, at least about 90%, about 95%, about 98%, about 99% or 100%, as compared to cardiac stem cells in the absence of an EphA2 receptor agonist. A skilled artisan can readily detect phosphorylation of Src kinases at tyrosine 416 and tyrosine 527 by western blot or immunostaining with antibodies against specific phosphorylation sites of Src kinase.

In alternative embodiment, the effective amount of EphA2 receptor agonist is determined by detecting an internalization of the ephrin A1/EphA2 complex in the treated cardiac stem cells. By way of an example only, the treated cardiac stem cells can be stained with ephrin A1 and EphA2 receptor antibodies using well-established immunostaining protocols, and then examined under a microscope, e.g., confocal microscope. The internalization of the ephrin A1/EphA2 complex can be detected, e.g., using a microscope, by a shift in distribution of the ephrin A1/EphA2 complex from plasma membrane to cytoplasm of treated cardiac stem cells.

In one embodiment, the effective amount of at least one EphA2 receptor agonist is sufficient to increase the locomotion speed of at least one cardiac stem cell within a myocardium by at least about 1.1-fold, at least about 1.2-fold, at least about 1.3-fold, at least about 1.4-fold or at least about 1.5 fold. In one embodiment, the effective amount of at least one EphA2 receptor agonist is sufficient to increase the locomotion speed of at least one cardiac stem cell within a myocardium by about 1.5-fold, about 2-fold or about 3-fold. In one embodiment, the effective amount of at least one EphA2 receptor agonist is sufficient to increase the locomotion speed of at least one cardiac stem cell by about 2-fold. Methods for measuring locomotion speed of cells within a myocardium, e.g., echocardiography, have been described in U.S. Pat. No. 7,547,674, the content of which is incorporated herein by reference in its entirety.

In one embodiment, the effective amount of EphA2 receptor agonist is about 0.1 ng/ml to about 400 ng/ml. In some embodiments, the effective amount of EphA2 receptor agonist can be present in an amount of about 25 ng/ml, about 50 ng/ml, about 75 ng/ml, about 100 ng/ml, about 125 ng/ml, about 150 ng/ml, about 175 ng/ml, about 200 ng/ml, about 225 ng/ml, about 250 ng/ml, about 275 ng/ml, about 300 ng/ml, about 325 ng/ml, about 350 ng/ml, about 375 ng/ml or about 400 ng/ml.

In one embodiment, the effective amount of EphA2 receptor is about 50 ng/ml to about 20 μg/mL. In another embodiment, the effective amount of EphA2 receptor agonist is about 200 ng/mL to about 1 μg/mL. In some embodiments, the effective amount of EphA2 receptor agonist can be present in an amount of about 75 ng/ml, about 100 ng/ml, about 150 ng/ml, about 200 ng/ml, about 250 ng/ml, about 300 ng/ml, about 350 ng/ml, about 400 ng/ml, about 450 ng/ml, about 500 ng/ml, about 550 ng/ml, about 600 ng/ml, about 650 ng/ml, about 700 ng/ml, about 750 ng/ml, about 800 ng/ml, about 850 ng/ml, about 900 ng/ml, about 950 ng/ml, or about 1 μg/mL. In some embodiments, the effective amount of EphA2 receptor agonist can be present in an amount of at least about 25 ng/ml, at least about 50 ng/ml, or at least about 100 ng/ml, at least about 250 ng/ml or at least about 500 ng/ml.

In one embodiment, the EphA2 receptor agonist is ephrin A1, or a variant thereof, e.g., ephrin A1-Fc. In such embodiment, the effective amount of ephrin A1, or a variant thereof, e.g., ephrin A1-Fc, is about 0.1 ng/ml to about 400 ng/ml. In some embodiments, the effective amount of ephrin A1, or a variant thereof, e.g., ephrin A1-Fc, can be present in an amount of about 25 ng/ml, about 50 ng/ml, about 75 ng/ml, about 100 ng/ml, about 125 ng/ml, about 150 ng/ml, about 175 ng/ml, about 200 ng/ml, about 225 ng/ml, about 250 ng/ml, about 275 ng/ml, about 300 ng/ml, about 325 ng/ml, about 350 ng/ml, about 375 ng/ml or about 400 ng/ml. In one embodiment, the effective amount of ephrin A1, or a variant thereof, e.g., ephrin A1-Fc, is about 50 ng/ml to about 20 μg/mL. In another embodiment, the effective amount of ephrin A1, or a variant thereof, e.g., ephrin A1-Fc, is about 200 ng/mL to about 1 μg/mL. In some embodiments, the effective amount of ephrin A1, or a variant thereof, e.g., ephrin A1-Fc, can be present in an amount of about 75 ng/ml, about 100 ng/ml, about 150 ng/ml, about 200 ng/ml, about 250 ng/ml, about 300 ng/ml, about 350 ng/ml, about 400 ng/ml, about 450 ng/ml, about 500 ng/ml, about 550 ng/ml, about 600 ng/ml, about 650 ng/ml, about 700 ng/ml, about 750 ng/ml, about 800 ng/ml, about 850 ng/ml, about 900 ng/ml, about 950 ng/ml, or about 1 μg/mL. In some embodiments, the effective amount of ephrin A1, or a variant thereof, e.g., ephrin A1-Fc, can be present in an amount of at least about 25 ng/ml, at least about 50 ng/ml, at least about 100 ng/ml, at least about 250 ng/ml or at least about 500 ng/ml.

Compositions of the Invention

Another aspect of the invention encompasses compositions comprising at least one cardiac stem cell described herein and an effective amount of at least one EphA2 receptor agonist described herein. In one embodiment, the composition further comprises at least one additional cytokine as described herein (e.g., IGF-1 and HGF).

In one embodiment, the composition further comprises a cell culture medium. As used herein, the term “cell culture medium” refers to any nutrient medium in which cardiac stem cells can be cultured in vitro. Examples of nutrients essential to cell metabolism and proliferation, e.g., amino acids, lipids, carbohydrates, vitamins and mineral salts can be included in the cell culture medium. In one embodiment, cell culture medium also comprises any substance essential to cell differentiation. One of skill in the art can determine an appropriate formulation of cell culture medium for culturing cardiac stem cells, based on the cell condition (e.g., morphology, viability, growth rate and cell density).

The composition of the invention can comprise a concentration of cardiac stem cells from about 2×10⁴ cells to about 2×10⁷ cells, about 1×10⁵ cells to about 6×10⁶ cells, or about 2×10⁶ cells. In one embodiment, the composition can comprise a concentration of at least about 1×10⁴ CSCs, at least about 5×10⁴ CSCs, at least about 1×10⁵ CSCs or at least about 1×10⁶ CSCs. In one embodiment, the composition can comprise a concentration of cardiac stem cells from about 1×10⁴ cells/ml to about 1×10⁸ cells/ml, or 1×10⁵ cells/ml to about 1×10⁷ cells/ml. In one embodiment, the composition can comprise a concentration of at least about 0.5×10⁴ CSCs per ml, at least about 5×10⁴ CSCs per ml, at least about 1×10⁵ CSCs per ml or at least about 1×10⁶ CSCs per ml. Depending on the use of compositions of the invention, a skilled artisan can determine an appropriate concentration of the cardiac stem cells in a composition. For example, for cell culture compositions, e.g., comprising a cell culture medium, lower concentrations of cardiac stem cells, e.g., 2×10⁴ cells-2×10⁵ cells can be selected for a culturing purpose. For therapeutic administration purpose, the composition of the invention can comprise higher concentrations of cardiac stem cells, e.g., about 1×10⁶ cells to about 2×10⁶ cells. The precise determination of an effective dose can be based on individual factors, including their size, age, size of the infarct, and amount of time since damage. Therefore, dosages can be readily adjusted for each individual patient by those skilled in the art.

For administration to a subject in need thereof, e.g., with a damaged myocardium, cardiac stem cells and EphA2 receptor agonist can be provided in a pharmaceutically acceptable composition. As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In one embodiment, the pharmaceutical acceptable composition can include a population of cardiac stem cells that further comprises vascular progenitor cells (VPCs) and myocyte progenitor cells (MPCs) in a particular ratio. This ratio can be adjusted to generate more vascular tissue (i.e. a higher number of VPCs compared to MPCs) or more myocardium (i.e. a higher number of MPCs compared to VPCs). The ratio of VPCs to MPCs in the pharmaceutical composition may be 1:20; 1:10; 1:5, 1:2; 1:1:2:1, 5:1; 10:1, and 20:1. In a preferred embodiment, the ratio of VPCs to MPCs is 1:1.

The pharmaceutically acceptable composition can further comprise one or more pharmaceutically carriers (additives) and/or diluents. As used herein, the term “pharmaceutically-acceptable carrier” means a pharmaceutically-acceptable material, composition or vehicle, such as a liquid, diluent, excipient, manufacturing aid or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include, but are not limited to, gelatin, buffering agents, such as magnesium hydroxide and aluminum hydroxide, pyrogen-free water, isotonic saline, Ringer's solution, pH buffered solutions, bulking agents such as polypeptides and amino acids, serum component such as serum albumin, HDL and LDL, and other non-toxic compatible substances employed in pharmaceutical formulations. Preservatives and antioxidants can also be present in the formulation. The terms such as “excipient”, “carrier”, “pharmaceutically acceptable carrier” or the like are used interchangeably herein.

Pharmaceutically acceptable carriers can vary in a composition of the invention, depending on the administration route and formulation. For example, the pharmaceutically acceptable composition of the invention can be delivered via injection. These routes for administration (delivery) include, but are not limited to, subcutaneous or parenteral including intravenous, intraarterial (e.g. intracoronary), intramuscular, intraperitoneal, intramyocardial, transendocardial, trans-epicardial, and infusion techniques. In one embodiment, the pharmaceutical acceptable composition is in a form that is suitable for myocardial injection. In another embodiment, the pharmaceutical composition is formulated for trans-epicardial or intracoronary injection.

When administering a pharmaceutical composition of the invention parenterally, it will be generally formulated in a unit dosage injectable form (solution, suspension, emulsion). The pharmaceutical formulations suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), and suitable mixtures thereof. In some embodiments, the pharmaceutical carrier can be a buffered solution (e.g. PBS), with or without an agent, such as an EphA2 receptor agonist or a cytokine described herein.

In some embodiments, the pharmaceutical composition can be formulated in an emulsion or a gel. In such embodiments, at least one cardiac stem cell can be encapsulated within a biocompatible gel, e.g., hydrogel and a peptide gel, which contains at least one EphA2 receptor agonist. The gel pharmaceutical composition can be implanted to the border zone of the damaged myocardium of a subject.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like.

The compositions can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation. With respect to compositions of the invention, however, any vehicle, diluent, or additive used should have to be biocompatible with cardiac stem cells.

The compositions can be isotonic, i.e., they can have the same osmotic pressure as blood and lacrimal fluid. The desired isotonicity of the compositions of the invention can be accomplished using sodium chloride, or other pharmaceutically acceptable agents such as dextrose, boric acid, sodium tartrate, propylene glycol or other inorganic or organic solutes. In one embodiment, sodium chloride is used in buffers containing sodium ions.

Viscosity of the compositions can be maintained at the selected level using a pharmaceutically acceptable thickening agent. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

In some embodiment, the compositions of the invention can be stored frozen. In such embodiments, an additive or preservative known for freezing cells can be included in the compositions. A suitable concentration of the preservative can vary from 0.02% to 2% based on the total weight although there may be appreciable variation depending upon the preservative or additive selected. One example of such additive or preservative can be dimethyl sulfoxide (DMSO) or any other cell-freezing agent known to a skilled artisan. In such embodiments, the composition will be thawed before use or administration to a subject.

Typically, any additives (in addition to the active cardiac stem cell(s), EphA2 receptor agonist(s) and/or cytokine(s)) can be present in an amount of 0.001 to 50 wt % solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %. For any therapeutic composition to be administered to a subject in need thereof, and for any particular method of administration, it is preferred to determine toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan.

Those skilled in the art will recognize that the components of the compositions should be selected to be biocompatible with respect to cardiac stem cells and chemically and/or biologically inert to EphA2 receptor agonists and/or optional cytokines. This will present no problem to those skilled in chemical and pharmaceutical principles, or problems can be readily avoided by reference to standard texts or by simple experiments (not involving undue experimentation).

The compositions of the invention can be prepared by mixing the ingredients following generally-accepted procedures. For example, isolated cardiac stem cells can be re-suspended in an appropriate pharmaceutically acceptable carrier and the mixture can be adjusted to the final concentration and viscosity by the addition of water or thickening agent and possibly a buffer to control pH or an additional solute to control tonicity. An effective amount of at least one EphA2 receptor described herein and any other additional cytokine can be mixed with the cell mixture. Generally the pH can vary from about 3 to about 7.5. In some embodiments, the pH of the composition can be about 6.5 to about 7.5. Compositions can be administered in dosages and by techniques well known to those skilled in the medical and veterinary arts taking into consideration such factors as the age, sex, weight, and condition of the particular patient, and the composition form used for administration (e.g., liquid). Dosages for humans or other mammals can be determined without undue experimentation by a skilled artisan.

Suitable regimes for initial administration and further doses or for sequential administrations can be varied. In one embodiment, a therapeutic regimen includes an initial administration followed by subsequent administrations, if necessary. In some embodiments, multiple administrations of cardiac stem cells can be injected to the subject's heart. For example, cardiac stem cells can be administered in two or more, three or more, four or more, five or more, or six or more injections. Injections can be made at the base of the heart, the apex, or the mid-region. In one embodiment, two injections of cardiac stem cells are administered at each of the apex, mid-region, and base. In one embodiment, more than one injection is administered in proximity to an area of a damaged heart tissue, e.g., the border of the area of the damaged heart tissue.

The subsequent injection can be administered immediately after the previous injection, or after at least about 1 minute, after at least about 2 minute, at least about 5 minutes, at least about 15 minutes, at least about 30 minutes, at least about 1 hour, at least about 2 hours, at least about 3 hours, at least about 6 hours, at least about 12 hours, at least about 24 hours, at least about 2 days, at least about 3 days, at least about 4 days, at least about 5 days, at least about 6 days or at least about 7 days. In some embodiments, the subsequent injection can be administered after at least about 1 week, at least about 2 weeks, at least about 1 month, at least about 2 years, at least about 3 years, at least about 6 years, or at least about 10 years.

After administration of the composition to a subject, at least one cardiac stem cell can translocate to the area of the damaged heart tissue of the subject after at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 1 day, at least about 2 days, at least about 3 days, at least about 1 week, at least about 2 weeks, or at least about 1 month. Methods for detection of translocation of cardiac stem cells have been described in U.S. Pat. No. 7,547,674, the content of which is incorporated herein by reference in its entirety.

In further embodiments, at least one cardiac stem cell translocated to the area of the damaged heart tissue can differentiate into a cardiomyocyte forming functional myocardium, thereby increasing cardiac function. Increased cardiac function can be reflected as increased exercise capacity, increased cardiac ejection volume, decreased left ventricular end diastolic pressure, decreased pulmonary capillary wedge pressure, increased cardiac output, increased cardiac index, lowered pulmonary artery pressures, decreased left ventricular end systolic and diastolic dimensions, decreased left and right ventricular wall stress, and decreased wall tension. Assessment tests for cardiac functions are well known to a skilled practitioner. Exemplary tests for cardiac function include, but not limited to, echocardiography, electrocardiogram, X-ray, magnetic resonance imaging, coronary catheterization, and heart CT scan. A skilled practitioner, e.g., cardiologist, can adjust the therapeutic number of cardiac stem cells and effective amount of EphA2 receptor agonist in each pharmaceutical composition and/or determine the need for subsequent administrations by performing various cardiac function tests known in the art on the subject who has received the composition of the invention, and comparing the test results to those measured in the same subject prior to administration. In some embodiments, a dosage comprising a composition of the invention is considered to be pharmaceutically effective if the dosage improves cardiac function, e.g., increased exercise capacity, by at least about 10%, at least about 15%, at least about 20%, at least about 30%, at least about 40%, or at least about 50%.

In one embodiment, the cardiac function is improved by more than 50%, e.g., at least about 60%, or at least about 70%. In another embodiment, the cardiac function is improved by at least about 80%, at least about 90% or greater, as compared to a control (e.g. in the absence of the composition described herein).

In some circumstances, one or more symptoms associated with cardiovascular diseases, e.g., heart failure, myocardial infarction, an age-related cardiomyopathy or a damaged myocardium, can be reduced or alleviated following administration of compositions of the invention. Symptoms of heart failure include, but are not limited to, fatigue, weakness, rapid or irregular heartbeat, dyspnea, persistent cough or wheezing, edema in the legs and feet, and swelling of the abdomen. Symptoms for myocardial infarction include, but are not limited to, prolonged chest pain, heart palpitations (i.e. abnormality of heartbeat), shortness of breath, and extreme sweating. Non-limiting symptoms of an age-related cardiomyopathy, e.g., restrictive cardiomyopathy, include coughing, difficulty breathing during normal activities or exercise, extreme fatigue, and swelling in the abdomen as well as the feet and ankles. In some embodiments, a dosage comprising a composition of the invention is considered to be pharmaceutically effective if the dosage alleviates at least one symptom of cardiovascular disease described above by at least about 10%, at least about 15%, at least about 20%, at least about 30%, at least about 40%, or at least about 50%. In one embodiment, at least one symptom is alleviated by more than 50%, e.g., at least about 60%, or at least about 70%. In another embodiment, at least one symptom is alleviated by at least about 80%, at least about 90% or greater, as compared to a control (e.g. in the absence of the composition described herein).

A further aspect of the invention relates to kits for treating at least one cardiac stem cell to be administered to as a subject in need thereof. In one embodiment, the kit comprises a composition of the invention, instruction for culturing the composition, and optionally cell culture supply, e.g., a cell culture flask, and/or cell culture medium, and/or at least one additional cytokine described herein. In another embodiment, the kit comprises a pharmaceutical composition, instructions for administering the pharmaceutical composition described herein, and optionally a delivery device and/or at least one additional agent, such as EphA2 receptor agonist or a cytokine described herein. The additional agent can be in the same pharmaceutical composition of the invention or they can be in separate pharmaceutical compositions packaged in different containers within the kit. The delivery devices that can be optionally included in the kit include a catheter, syringe, or any other appropriate delivery device.

Selection of Subjects in Need Thereof

Yet another aspect of the invention relates to the use of methods, compositions and kits described herein to increase motility of cardiac stem cells to be administered to a subject in need thereof. The inventors have demonstrated that increasing translocation of cardiac stem cells with an EphA2 receptor agonist, e.g., ephrin A1 or ephrin A1-Fc, from the injection site to the infarcted area enhances myocardium regeneration in an in vivo mouse model, as compared to in the absence of the EphA2 receptor agonist.

Accordingly, methods, compositions and kits of the invention can be used for treatment of cardiovascular disease, including, but not limited to, atherosclerosis, ischemia, hypertension, restenosis, angina pectoris, rheumatic heart disease, congenital cardiovascular defects, age-related cardiomyopathy, and arterial inflammation and other disease of the arteries, arterioles and capillaries. In one embodiment, the methods, compositions and kits of the invention provide for the repair and/or regeneration of a damaged myocardium resulting from one of the diseases listed above or from the general decline of myocardial cells with age.

The terms “treatment” and “treating” as used herein, with respect to treatment of a disease, means preventing the progression of the disease, or altering the course of the disorder (for example, but are not limited to, slowing the progression of the disorder), or reversing a symptom of the disorder or reducing one or more symptoms and/or one or more biochemical markers in a subject, preventing one or more symptoms from worsening or progressing, promoting recovery or improving prognosis. For example, in the case of treating cardiovascular disease, e.g., myocardial infarction, therapeutic treatment refers to improved cardiac function described herein after administration of the composition of the invention. In another embodiment, the therapeutic treatment refers to alleviation of at least one symptom associated with cardiovascular disease, e.g., myocardial infarction. Measurable lessening includes any statistically significant decline in a measurable marker or symptom, such as measuring cardiac biomarkers, such as cardiac troponin I in the blood, assessing the swelling in the arm or leg, or assessing the cardiac function with electrophysiological tests such as echocardiography (as described in detail below) after treatment. In one embodiment, at least one symptom of cardiovascular disease, e.g., myocardial infarction, is alleviated by at least about 10%, at least about 15%, at least about 20%, at least about 30%, at least about 40%, or at least about 50%. In another embodiment, at least one symptom is alleviated by more than 50%, e.g., at least about 60%, or at least about 70%. In one embodiment, at least one symptom is alleviated by at least about 80%, at least about 90% or greater, as compared to a control (e.g. in the absence of the composition described herein).

In one embodiment, subjects in need thereof are selected prior to administering the compositions or kits of the invention or employing the methods described herein. In some embodiments, the subject in need thereof can be diagnosed with or suffering from a damaged myocardium. In another embodiment, the subject in need thereof can be diagnosed with or suffering from a myocardial infarction. In certain embodiment, the subject in need thereof can be diagnosed with or suffering a heart failure. In some embodiments, the subject in need thereof can be diagnosed with or suffering from an age-related cardiomyopathy. As used herein, the term “age-related cardiomyopathy” refers to the deterioration of the myocardium (heart muscle tissue) as a result of intrinsic mechanisms occurring as a subject ages. An example of age-related cardiomyopathy is restrictive cardiomyopathy.

A set of guidelines for diagnosis of heart diseases, e.g., myocardial infarction, has been proposed jointly by the American college of Cardiologists and the Europe School of Cardiology (ACC/ESC; Alpert et al. 2000). These guidelines emphasize the importance of changes in the levels of the biochemical markers cardiac troponin I (cTnI) and creatine kinase MB form (CK-MB), in combination with other diagnostic factors such as electrocardiogram (ECG) results, especially ST segment elevation, but also ST segment depression or T-wave inversion, and typical symptoms of severe chest pain and dyspnoea.

To diagnose for ischemic heart disease in a subject, an electrocardiogram can be performed. An electrocardiogram (ECG) is a recording of the electrical activity of the heart. Abnormalities in the electrical activity usually occur with heart attacks and can identify the areas of heart muscle that are deprived of oxygen and/or areas of muscle that have died. In some patients, the diagnosis can be made through detection of elevated cardiac enzymes in the blood. Cardiac enzymes are proteins that are released into the blood by dying heart muscles. These cardiac enzymes are creatine phosphokinase (CPK), special sub-fractions of CPK (specifically, the MB fraction of CPK), and troponin, and their levels can be measured in blood. These cardiac enzymes typically are elevated in the blood several hours after the onset of a heart attack. A series of blood tests for the enzymes performed over a 24-hour period are useful not only in confirming the diagnosis of heart attack, but the changes in their levels over time also correlates with the amount of heart muscle that has died. The B-type of natriuretic peptide (BNP) together with pro-BNP, NT-proBP (EP1363128, EP1666881) has also proven to be a further effective biochemical marker in myocardial diagnostics. Other biomarkers that can be used for diagnosis of heart diseases, e.g., myocardial infarction, include, but are not limited to, the ones disclosed in US Pat. App. Pub. Nos: US 2009/0208986, US 2010/0151504 and PCT App. No.: WO 2006/120391, the contents of which are incorporated herein by its entirety.

Heart diseases can be diagnosed with any methods known to a skilled practitioner, e.g., chest X-ray, or monitoring heart rate, blood pressure and electrocardiogram during exercise stress test. Other tests for diagnosis of heart disease include, but are not limited to, an echocardiogram that uses ultrasound to evaluate one's heart muscle, heart valves, and risk for heart disease; cardiac catheterization (also called cardiac cath or coronary angiogram) that allows a physician to “see” how well one's heart is functioning; an electrophysiology (EP) study that records the electrical activity and the electrical pathways of one's heart; cardiac computed tomography (CT) that uses CT technology with or without intravenous (IV) contrast (dye) to visualize the heart anatomy, coronary circulation, and great vessels (which includes the aorta, pulmonary veins, and arteries); a heart biopsy (also called myocardial biopsy or cardiac biopsy) that involves using a bioptome (a small catheter with a grasping device on the end) to obtain a small piece of heart muscle tissue that is sent to a laboratory for analysis; and MRI (magnetic resonance imaging) that obtains information about the heart as it is beating, creating images of the heart throughout its pumping cycle. Other imaging methods, e.g., magnetic resonance imaging, for diagnosis of heart diseases disclosed in U.S. Pat. No. 3,951,140, U.S. Pat. No. 6,205,349 and U.S. Pat. No. 4,867,963 (the contents of which are incorporated herein by their entirety), are also included within the scope of the invention.

In some embodiments, the subject selected for the methods described herein can be previously diagnosed with a damaged myocardium and is now recovered. In other embodiments, the subject selected for the methods described herein can have undergone other cardiac interventions.

As used herein, a “subject” can mean a human or an animal. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. A patient or a subject includes any subset of the foregoing, e.g., all of the above, or includes one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

In one embodiment, the subject is a mammal. The mammal can be a human, non-human primate, mouse, rat, dog, cat, horse, or cow, but are not limited to these examples. Mammals other than humans can be advantageously used as subjects that represent animal models of stem cell therapy for repair for damaged myocardium. In addition, the methods and compositions described herein can be employed in domesticated animals and/or pets.

Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the paragraphed invention, because the scope of the invention is limited only by the paragraphs. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type described herein and/or which will become apparent to those persons skilled in the art upon reading this disclosure and so forth.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, the term “administer” or “administration” refers to the placement of a composition into a subject by a method or route which results in at least partial localization of the composition at a desired site such that desired effect is produced. Routes of administration suitable for the methods of the invention include, but are not limited to, injection and delivery by a catheter. Generally, local administration results in more of the composition being delivered to a specific location as compared to the entire body of the subject.

The term “hydrogel” as used herein refers to natural or synthetic polymers that show superabsorbent properties (having even over 99% water) and possess a degree of flexibility similar to natural tissue, due to their significant water content. Examples of hydrogels used as scaffolds in tissue engineering or reservoirs in local drug delivery include, but are not limited to, methylcellulose, hylaronan, and other naturally derived polymers. In one embodiment, the hydrogel is biodegradable.

The term “translocate” or “translocation” as used herein, with respect to movement of cardiac stem cells, refers to changing from one place or position to another. The terms “translocation” or “locomotion” are used herein interchangeably.

The term “increase” or “enhance” as used herein generally means an increase by a statistically significant amount. In one embodiment, “increase” or “enhance” refers to an increase by at least 10% as compared to a reference level, for example an increase by at least about 20%, or at least about 30%, or at least about 40%, or at least about 50%, or at least about 60%, or at least about 70%, or at least about 80%, or at least about 90% or up to and including a 100% increase, or any increase between 10-100% as compared to a reference level. The reference level as used herein refers to a control in the absence of, e.g., a, EphA2 receptor agonist. In one embodiment, the reference level is measured prior to administration of the composition described herein.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

The present invention may be defined in any of the following numbered paragraphs:
1. A method of treating cardiac stem cells to be administered to a subject in need thereof, comprising:
   a. contacting a population of cardiac stem cells with an effective amount of at least one EphA2 receptor agonist; and
   b. administering the population of cardiac stem cells from step (a) to the subject in need thereof.
2. The method of paragraph 1, wherein the at least one EphA2 receptor agonist is ephrin A1, or a variant thereof.
3. The method of any of paragraphs 1-2, wherein the at least one EphA2 receptor agonist is ephrin A1-Fc, or a variant thereof.
4. The method of any of paragraphs 1-3, wherein the population of cardiac stem cells is administered in spatial proximity to an area of a damaged heart tissue of the subject.
5. The method of any of paragraphs 1-4, wherein the population of cardiac stem cells is administered to the border of the area of the damaged heart tissue of the subject.
6. The method of any of paragraphs 1-5, wherein the population of cardiac stem cells is administered by injection.
7. The method of any of paragraphs 1-6, wherein the population of cardiac stem cells is administered by a catheter.
8. The method of any of paragraphs 1-7, wherein the effective amount is sufficient to increase motility of the cardiac stem cells by at least about 10%, as compared to cardiac stem cells in the absence of an EphA2 receptor agonist.
9. The method of any of paragraphs 1-8, wherein the effective amount is sufficient to induce rearrangement of actin cytoskeleton from a sessile to a motile state, as compared to cardiac stem cells in the absence of an EphA2 receptor agonist.
10. The method of any of paragraphs 1-9, wherein the effective amount is about 50 ng/ml to about 20 μg/ml.
11. The method of any of paragraphs 1-10, wherein the effective amount is about 200 ng/ml to about 1 μg/ml.
12. The method of any of paragraphs 1-11, wherein the cardiac stem cells are contacted with at least one EphA2 receptor agonist for about 5 to about 30 minutes.
13. The method of any of paragraphs 1-12, wherein the cardiac stem cells are contacted with at least one EphA2 receptor agonist for about 15 minutes.
14. The method of any of paragraphs 1-13, wherein the subject in need thereof is diagnosed with or suffering from a myocardial infarction.
15. The method of any of paragraphs 1-14, wherein the subject in need thereof is diagnosed with or suffering from a heart failure.
16. The method of any of paragraphs 1-15, wherein the subject in need thereof is diagnosed with or suffering from an age-related cardiomyopathy.
17. The method of any of paragraphs 1-16, wherein the subject is a mammal.
18. The method of paragraph 17, wherein the mammal is a human.
19. The method of any of paragraphs 1-18, wherein the cardiac stem cells are isolated from a myocardial tissue of the subject.
20. A composition comprising at least one cardiac stem cell and an effective amount of at least one EphA2 receptor agonist.
21. The composition of paragraph 20, further comprising a pharmaceutically acceptable carrier.
22. The composition of paragraph 20, further comprising a cell culture medium.
23. The composition of any of paragraphs 20-22, wherein the at least one EphA2 receptor agonist is ephrin A1, or a variant thereof.
24. The composition of any of paragraphs 20-23, wherein the at least one EphA2 receptor agonist is ephrin A1-Fc, or a variant thereof.
25. The composition of any of paragraphs 20-24, wherein the effective amount is sufficient to increase motility of the cardiac stem cells by at least about 10%, as compared to cardiac stem cells in the absence of an EphA2 receptor agonist.
26. The composition of any of paragraphs 20-25, wherein the effective amount is sufficient to increase internalization of EphA2 receptor by at least about 10%, as compared to cardiac stem cells in the absence of an EphA2 receptor agonist.
27. The composition of any of paragraphs 20-26, wherein the effective amount is about 50 ng/ml to about 20 μg/ml.
28. The composition of any of paragraphs 20-27, wherein the effective amount is about 200 ng/ml to about 1 μg/m1.
29. The composition of any of paragraphs 20-28, wherein the cardiac stem cell is isolated from a myocardial tissue of a subject.
30. Use of the composition of paragraph 20 for administration to a subject in need thereof.
31. The use of paragraph 30, wherein the subject in need thereof is diagnosed with or suffering from a myocardial infarction.
32. The use of any of paragraphs 30-31, wherein the subject in need thereof is diagnosed with or suffering from a heart failure.
33. The use of any of paragraphs 30-32, wherein the subject in need thereof is diagnosed with or suffering from an age-related cardiomyopathy.
34. The use of any of paragraphs 30-33, wherein the subject is a mammal.
35. The use of paragraph 34, wherein the mammal is a human.
36. The use of any of paragraphs 30-35, wherein the composition is administered in spatial proximity to an area of a damaged heart tissue of the subject.
37. The use of any of paragraphs 30-36, wherein the composition is administered to the border of the area of the damaged heart tissue of the subject.
38. The use of any of paragraphs 30-37, wherein the administration is performed by injection.
39. The use of any of paragraphs 30-38, wherein the administration is performed via a catheter.

### EXAMPLES

The examples presented herein relate to stimulation of cardiac stem cells (CSCs) with an EphA2 receptor agonist, e.g., ephrin A1 ligand, for enhancing migration of CSCs to the infracted myocardium. In one embodiment, the ephrin A1 ligand is administered in the border zone of an infracted heart to recruit resident CSCs to the site of the myocardial injury for myocardial regeneration. In another embodiment, exogenous CSCs can be pre-treated with ephrin A1 ligand or a variant thereof, e.g., ephrin A1-Fc, prior to delivery to the border zone of an infarcted heart for repairing the damaged myocardium in vivo.

Accordingly, the methods and EphA2 receptor agonists, e.g., ephrin A1 or a variant thereof described herein, can be used for treatment of heart diseases, such as myocardial infarction. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the paragraphs to the invention, but are rather intended to be exemplary of certain embodiments. Any variations in the exemplified methods which occur to the skilled artisan are intended to fall within the scope of the present invention.

### Example 1

### Migration of Human Cardiac Stem Cells (hCSCs) in the Infarcted Mouse Heart

One of the major factors responsible for successful implementation of cell therapy in the diseased heart involves the migratory ability of the delivered cardiac stem cells (CSCs). The possibility exists that the mechanisms that govern the egress of CSCs from their niches are also implicated in the translocation of CSCs to the injured myocardium. The inventors have determined that stem cell niches in the mouse heart are composed of CSCs expressing the EphA2 receptor and myocytes displaying the ephrin A1 ligand. It was next sought to assess whether the EphA2 receptor-ephrin A1 ligand system is implicated in the motility of human CSCs (hCSCs) in vivo. The hCSCs expressed c-kit and were negative for hematopoietic markers including CD34, CD45, CD133, CD105, CD90 and multiple markers of bone marrow cell lineages. They were also negative for cardiac transcription factors (Nkx2.5, MEF2C, GATA4, GATA6, Etsl) and cardiac cytoplasmic/membrane proteins (α-sarcomeric actin, a-smooth muscle actin, vWf, CD31). EGFP-tagged hCSCs were pre-treated for 15 minutes with ephrin A1 and subsequently injected in the border zone of the infarcted mouse heart acutely after infarction. Two days later, the translocation of EGFP-positive hCSCs to the necrotic myocardium was measured quantitatively by confocal microscopy. Ephrin A1-activated hCSCs showed a polarized morphology and were aligned in proximity and within the infarct. Conversely, untreated hCSCs preserved a round shape and were confined to the site of injection. Treatment with recombinant ephrin A1 promoted internalization of EphA2 receptor, enhanced actin bundling and increased the spontaneous motility of hCSCs. Moreover, EphA2 activation by ephrin A1 potentiated the chemotactic response of hCSCs. The differential expression of EphA2 and ephrin A1 in CSCs and myocytes, respectively, was confirmed at the RNA and protein level by quantitative RT-PCR, Western blotting and immunocytochemistry. EphA2, an established regulator of cell adhesion and chemotaxis, was detected in hCSCs while its ligand ephrin A1 was restricted to human cardiomyocytes. In the absence of ephrin A1 treatment, serially passaged hCSCs synthesized ephrin A1 which accumulated at the cell trailing edge. However, senescent hCSCs showed negligible levels of ephrin A1 and failed to acquire a polarized morphology. In conclusion, signaling by EphA2/ephrin A1 favors hCSC motility, mediating their migration to areas of injury. Without wishing to be bound by theory, in situ activation of resident hCSCs with ephrin A1 or their ex vivo manipulation prior to delivery to the myocardium can improve cell targeting to sites of damage, providing a novel strategy for the treatment of heart failure.

### Example 2

### Expression of EphA2 and Ephrin A1 in the Myocardium

C-kit positive human cardiac stem cells (hCSCs) are organized in niches which are located preferentially in the atria and apex. hCSCs are functionally connected to cardiomyocytes, which act as supporting cells and influence the fate of adjacent primitive cells. The components of this cell-to-cell interaction within the cardiac niches are largely unknown. While the effect of ephrin/Eph system on cell motility has been previously studied in other self-renewing organs, the effect of the ephrin/Eph system on the motility of resident stem cells within the myocardium was an unknown. Therefore, it was first sought to determine the presence of the ephrin/Eph family members in hCSCs within the myocardium. The presence of the ephrin/Eph family members was measured by quantitative real time-polymerase chain reaction (qRT-PCR) in hCSCs and human cardiomyocytes to search for gene products differentially expressed in these two cell classes. EphA2 receptor mRNA was abundant in hCSCs, while transcripts of the ephrin A1 ligand were highly represented in cardiomyocytes (Data not shown). A similar distribution was found in mouse CSCs and myocytes. The preferential localization of EphA2 in hCSCs and ephrin A1 in human cardiomyocytes was confirmed by Western blotting (Data not shown) and immunolabeling (Data not shown).

To establish whether these findings in isolated hCSCs and myocytes mimicked the tissue properties, the expression of EphA2 and ephrin A1 was determined in the human myocardium. Clusters of hCSCs were nested in the interstitium and coupled with neighboring myocytes by connexin 43 and N-cadherin (Data not shown). The ephrin A1 ligand was present in myocytes adjacent to EphA2-positive hCSCs (Data not shown). Importantly, ephrin A1 was restricted to the myocyte compartment; it was not detected in endothelial cells (ECs), smooth muscle cells (SMCs) and fibroblasts (Data not shown). These data raise that possibility that cardiomyocytes carrying the ephrin A1 ligand interact with hCSCs possessing the EphA2 receptor and, as a result, modify their motile phenotype within the cardiac niches.

### Example 3

### Ephrin A1 Potentiates hCSC Motility

Cardiomyocytes may influence the behavior of hCSCs by secretion of a soluble signal or direct cell-to-cell contact. To test these possibilities, the functional role of the ephrin A1-EphA2 axis was established in vitro by exposing EphA2-positive hCSCs to a human ephrin A1-Fcy chimeric protein (ephrin A1-Fc), or control human IgG (Fc). The rapid adhesion of hCSCs to ephrin A1-coated surfaces documented the functional competence of the EphA2 receptor (Data not shown). Treatment with ephrin A1-Fc promoted the rearrangement of the actin cytoskeleton changing the shape of hCSCs from a sessile to a motile state (Data not shown). Ephrin A1 resulted in rapid internalization of the ephrin A1/EphA2 complex from the plasma-membrane to the cytoplasm (Data not shown) and the accumulation of EphA2 at the leading edge of migrating hCSCs (Data not shown).

Since Src kinase represents a well-established downstream effector of Eph receptor signaling, the state of phosphorylation of Src was evaluated in ephrin A1-stimulated hCSCs. Following ligand binding, there was a time-dependent increase in the phosphorylation of the activatory site of Src kinase at tyrosine 416, which was coupled with a concomitant decrease in phosphorylation of the inhibitory site of Src at tyrosine 527 (Data not shown). Thus, these post-translational modifications of Src kinase indicate that the EphA2 pathway was activated in hCSCs by ephrin A1.

The chemoattractant hepatocyte growth factor (HGF) favors the migration of stem cells to sites of ischemic myocardial damage. As a positive control, hCSCs moved towards HGF in a Transwell migration assay (Data not shown). Importantly, pre-stimulation of hCSCs with recombinant ephrin A1-Fc enhanced the spontaneous motility and the chemotactic responses of these cells to low concentrations of HGF. However, the additive effect of ephrin A1 on cell locomotion was no longer apparent when hCSCs were exposed to high quantities of HGF, which saturated the migratory machinery of the cells. In addition, ephrin A1 did not affect the rate of proliferation and apoptosis of hCSCs (Data not shown). Collectively, these findings demonstrate the critical role of the ephrin A1/EphA2 system in the migratory ability of hCSCs.

### Example 4

### Migration of Endogenous Stem Cells to the Infarcted Myocardium

The in vitro results in Example 3 raised the possibility that the ephrin A1-EphA2 axis enhances the migration of endogenous stem cells to the injured myocardium, promoting the recovery of structure and function of the infarcted heart. Two days after coronary artery occlusion in the mouse, the expression of ephrin A1 markedly increased in the border zone and distant myocardium (Data not shown). Similarly, ephrin A1 was determined to be up-regulated in human myocytes from hearts with ischemic cardiomyopathy (Data not shown). These results suggest that the synthesis of this ephrin A1 ligand by the cardiac muscle compartment constitutes an adaptive response aiming at the recruitment of hCSCs at sites of tissue damage. However, this regenerative process is predominantly restricted to the surviving region of the ventricular wall, and thus contributes minimally to the repair of the infarcted heart.

To determine whether ephrin A1 positively affects the motility of EphA2-positive CSCs in vivo, a transgenic mouse model in which the expression of enhanced green fluorescent protein (EGFP) is driven by the c-kit promoter was employed. Ephrin A1-Fc was administered in the border zone of acutely infarcted mice and the number of CSCs present in proximity of the necrotic tissue was measured 2 days later. Infarcted mice injected with human IgG (Fc) were used as controls. In comparison with Fc-treated mice, the intramyocardial delivery of ephrin A1-Fc resulted in a two-fold increase in the number of c-kit-EGFP-positive CSCs (Data not shown).

The accumulation of stem cells in the presence of ephrin A1 may involve enhanced recruitment, increased stem cell division, reduced apoptosis, or a combination of these variables. However, the fraction of cycling Ki-67-positive CSCs was determined and determined to be comparable in ephrin-A1-Fc and Fc infarcted hearts. Similarly, apoptosis of CSCs, measured by the terminal deoxynucleotidyl transferase (TdT) assay, did not differ with ephrin A1 or its absence (Data not shown). Hence, ephrin A1 favors the translocation and homing of CSCs at the site of myocardial injury, potentiating the cellular responses that mediate the repair of the infarcted heart.

### Example 5

### Pre-treatment of hCSCs with Ephrin A1 Enhances hCSC Motility In Vivo

The finding that intramyocardial delivery of ephrin A1-Fc enhances accumulation of CSCs in proximity of the necrotic tissue in the mouse heart pointed to a potential clinical implication of ephrin A1 and the activation of EphA2 in hCSCs as a mechanism of myocardial regeneration in humans. To assess this possibility, hCSCs were exposed to ephrin A1 in vitro prior to their delivery to the border zone of infarcted immunosuppressed mice. Initially, the movement of hCSCs from the site of injection to the infarcted myocardium was monitored by two-photon microscopy and live imaging.

With respect to untreated hCSCs, ephrin A1-treated hCSCs showed a two-fold increase in the speed of locomotion within the myocardium (Data not shown). A greater displacement in the trajectory of migrating hCSCs within the tissue was also recognized (Data not shown). The enhanced movement of hCSC with ephrin A1 was associated with a significantly increased number of migrating cells (Data not shown), indicating that the ephrinAl/EphA2 pathway positively influenced the timing and degree of hCSC trafficking and, therefore, the onset and potentially the extent of the cardiac repair process.

### Example 6

### Myocardial Regeneration by hCSCs Pre-Treated with Ephrin A1

Enhanced homing of hCSCs to the ischemic region of the left ventricle (LV) may favorably affect the magnitude of myocardial region. Infarcted rats treated with control or ephrin A1-treated hCSCs were sacrificed two weeks after surgery and cell implantation. In all hearts from both groups, infarct size involved an average 30% loss of left ventricular (LV) myocytes (Data not shown). Myocardial regeneration, with extensive replacement of the necrotic myocardium, was detected in both groups injected with control or ephrin A1-treated hCSCs (Data not shown). The areas of cardiac repair consisted of clusters of closely packed human cardiomyocytes and coronary vessels. Although cardiac repair was detected in infarcted hearts treated with control or ephrin A1-treated hCSCs, the aggregate volume of the regenerated myocyte mass was 2-fold greater in animals injected with ephrin A1-tretead hCSCs than with control cells. The volume of newly formed myocytes in the two groups of animals was comparable, indicating that an increase in myocyte number was responsible for the greater degree of myocardial regeneration in rats treated with ephrin A1-stimulated hCSCs.

SEQUENCES

| | |
|---|---|
| 1 | MEFLWAPLLG LCCSLAAADR HTVFWNSSNP KFRNEDYTIH VQLNDYVDII CPHYEDHSVA |
| 61 | DAAMEQYILY LVEHEEYQLC QPQSKDQVRW QCNRPSAKHG PEKLSEKFQR FTPFTLGKEF |
| 121 | KEGHSYYYIS KPIHQHEDRC LRLKVTVSGK ITHSPQAHDN PQEKRLAADD PEVRVLHSIG |
| 181 | HSAAPRLFPL AWTVLLLPLL LLQTP (SEQ ID NO: 1) |
| 1 | MEFLWAPLLG LCCSLAAADR HTVFWNSSNP KFRNEDYTIH VQLNDYVDII CPHYEDHSVA |
| 61 | DAAMEQYILY LVEHEEYQLC QPQSKDQVRW QCNRPSAKHG PEKLSEKFQR FTPFTLGKEF |
| 121 | KEGHSYYYIS HSPQAHDNPQ EKRLAADDPE VRVLHSIGHS AAPRLFPLAW TVLLLPLLLL |
| 181 | QTP (SEQ ID NO: 2) |
| 1 | MELQAARACF ALLWGCALAA AAAAQGKEVV LLDFAAAGGE LGWLTHPYGK GWDLMQNIMN |
| 61 | DMPIYMYSVC NVMSGDQDNW LRTNWVYRGE AERIFIELKF TVRDCNSFPG GASSCKETFN |
| 121 | LYYAESDLDY GTNFQKRLFT KIDTIAPDEI TVSSDFEARH VKLNVEERSV GPLTRKGFYL |
| 181 | AFQDIGACVA LLSVRVYYKK CPELLQGLAH FPETIAGSDA PSLATVAGTC VDHAVVPPGG |
| 241 | EEPRMHCAVD GEWLVPIGQC LCQAGYEKVE DACQACSPGF FKFEASESPC LECPEHTLPS |
| 301 | PEGATSCECE EGFFRAPQDP ASMPCTRPPS APHYLTAVGM GAKVELRWTP PQDSGGREDI |
| 361 | VYSVTCEQCW PESGECGPCE ASVRYSEPPH GLTRTSVTVS DLEPHMNYTF TVEARNGVSG |
| 421 | LVTSRSFRTA SVSINQTEPP KVRLEGRSTT SLSVSWSIPP PQQSRVWKYE VTYRKKGDSN |
| 481 | SYNVRRTEGF SVTLDDLAPD TTYLVQVQAL TQEGQGAGSK VHEFQTLSPE GSGNLAVIGG |
| 541 | VAVGVVLLLV LAGVGFFIHR RRKNQRARQS PEDVYFSKSE QLKPLKTYVD PHTYEDPNQA |
| 601 | VLKFTTEIHP SCVTRQKVIG AGEFGEVYKG MLKTSSGKKE VPVAIKTLKA GYTEKQRVDF |
| 661 | LGEAGIMGQF SHHNIIRLEG VISKYKPMMI ITEYMENGAL DKFLREKDGE FSVLQLVGML |
| 721 | RGIAAGMKYL ANMNYVHRDL AARNILVNSN LVCKVSDFGL SRVLEDDPEA TYTTSGGKIP |
| 781 | IRWTAPEAIS YRKFTSASDV WSFGIVMWEV MTYGERPYWE LSNHEVMKAI NDGFRLPTPM |
| 841 | DCPSAIYQLM MQCWQQERAR RPKFADIVSI LDKLIRAPDS LKTLADFDPR VSIRLPSTSG |
| 901 | SEGVPFRTVS EWLESIKMQQ YTEHFMAAGY TAIEKVVQMT NDDIKRIGVR LPGHQKRIAY |
| 961 | SLLGLKDQVN TVGIPI (SEQ ID NO: 3) |

It is understood that the foregoing detailed description and examples are illustrative only and are not to be taken as limitations upon the scope of the invention. Various changes and modifications to the disclosed embodiments, which will be apparent to those of skill in the art, may be made without departing from the spirit and scope of the present invention. Further, all patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating cardiac stem cells to be administered to a subject in need thereof, comprising:
a. contacting a population of cardiac stem cells with an effective amount of at least one EphA2 receptor agonist; and
b. administering the population of cardiac stem cells from step (a) to the subject in need thereof.

2. The method of claim 1, wherein the at least one EphA2 receptor agonist is ephrin A1, ephrin A1-Fc, or a variant thereof, or a combination thereof.

3. (canceled)

4. The method of claim 1, wherein the population of cardiac stem cells is administered in spatial proximity to an area of a damaged heart tissue of the subject, or is administered to the border of the area of the damaged heart tissue of the subject.

5. (canceled)

6. The method of claim 1, wherein the population of cardiac stem cells is administered by injection, a catheter, or a combination thereof.

7. (canceled)

8. The method of claim 1, wherein the effective amount is sufficient to increase motility of the cardiac stem cells by at least about 10%, as compared to cardiac stem cells in the absence of an EphA2 receptor agonist, or is sufficient to induce rearrangement of actin cytoskeleton from a sessile to a motile state, as compared to cardiac stem cells in the absence of an EphA2 receptor agonist.

9. (canceled)

10. The method of claim 1, wherein the effective amount is about 50 ng/ml to about 20 μg/ml, or about 200 ng/ml to about 1 μg/ml.

11. (canceled)

12. The method of claim 1, wherein the cardiac stem cells are contacted with at least one EphA2 receptor agonist for about 5 to about 30 minutes, or for about 15 minutes.

13. (canceled)

14. The method of claim 1, wherein the subject in need thereof is diagnosed with or suffering from a myocardial infarction, a heart failure, or an age-related cardiomyopathy.

15. (canceled)

16. (canceled)

17. The method of claim 1, wherein the subject is a mammal.

18. The method of claim 17, wherein the mammal is a human.

19. The method of claim 1, wherein the cardiac stem cells are isolated from a myocardial tissue of the subject.

20. A composition comprising at least one cardiac stem cell and an effective amount of at least one EphA2 receptor agonist.

21. The composition of claim 20, further comprising a pharmaceutically acceptable carrier, or a cell culture medium.

22. (canceled)

23. The composition of claim 20, wherein the at least one EphA2 receptor agonist is ephrin A1, ephrin A1-Fc, or a variant thereof, or a combination thereof.

24. (canceled)

25. The composition of claim 20, wherein the effective amount is sufficient to increase motility of the cardiac stem cells by at least about 10%, as compared to cardiac stem cells in the absence of an EphA2 receptor agonist, or is sufficient to increase internalization of EphA2 receptor by at least about 10%, as compared to cardiac stem cells in the absence of an EphA2 receptor agonist.

26. (canceled)

27. The composition of claim 20, wherein the effective amount is about 50 ng/ml to about 20 μg/ml, or about 200 ng/ml to about 1 μg/ml.

28. (canceled)

29. The composition of claim 20, wherein the cardiac stem cell is isolated from a myocardial tissue of a subject.

30. The composition of claim 20, wherein the composition is used for administration to a subject in need thereof.

31. The composition of claim 30, wherein the subject in need thereof is diagnosed with or suffering from a myocardial infarction, a heart failure or an age-related cardiomyopathy.

32. (canceled)

33. (canceled)

34. (canceled)

35. (canceled)

36. (canceled)

37. (canceled)

38. The composition of claim 20, wherein the composition is injectable.

39. (canceled)


SEQUENCE LISTING (3 sequences)

SEQ ID NO: 1 - protein, 205 aa, Homo sapiens
MEFLWAPLLGLCCSLAAADRHTVFWNSSNPKFRNEDYTIHVQLNDYVDIICPHYEDHSVADAAMEQYILYLVEHEEYQLCQPQSKDQVRWQCNRPSAKHGPEKLSEKFQRFTPFTLGKEFKEGHSYYYISKPIHQHEDRCLRLKVTVSGKITHSPQAHDNPQEKRLAADDPEVRVLHSIGHSAAPRLFPLAWTVLLLPLLLLQTP

SEQ ID NO: 2 - protein, 183 aa, Homo sapiens
MEFLWAPLLGLCCSLAAADRHTVFWNSSNPKFRNEDYTIHVQLNDYVDIICPHYEDHSVADAAMEQYILYLVEHEEYQLCQPQSKDQVRWQCNRPSAKHGPEKLSEKFQRFTPFTLGKEFKEGHSYYYISHSPQAHDNPQEKRLAADDPEVRVLHSIGHSAAPRLFPLAWTVLLLPLLLLQTP

SEQ ID NO: 3 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI